(19) United States
(12) Patent Application Publication
(10) Pub. No.: US 2022/0356216 A1
(43) Pub. Date: Nov. 10, 2022

(54) LIGHT-GENERATING FUSION PROTEINS CAPABLE OF SELF-ACTIVATION

(71) Applicant: Technion Research & Development Foundation Limited, Haifa (IL)

(72) Inventors: Avraham SCHROEDER, Haifa (IL); Omer ADIR, Haifa (IL)

(73) Assignee: Technion Research & Development Foundation Limited, Haifa (IL)

(21) Appl. No.: 17/737,044

(22) Filed: May 5, 2022

Related U.S. Application Data
(60) Provisional application No. 63/184,876, filed on May 6, 2021.

(51) Int. Cl. C07K 14/435; A61K 9/127

(52) U.S. Cl. CPC C07K 14/43595; A61K 9/1272; A61K 38/00

(57) ABSTRACT

A composition of matter comprising a fusion protein attached to the outer surface of a substrate is disclosed. The fusion protein comprises a light-generating protein and a light-transducing protein. Uses of the composition of matter are disclosed.

### RELATED APPLICATION(S)

This application claims the benefit of priority under 35 USC § 119(e) of U.S. Provisional Patent Application No. 63/184,876 filed on May 6, 2021, the contents of which are all incorporated by reference as if fully set forth herein in their entirety.

### SEQUENCE LISTING STATEMENT

The ASCII file, entitled 92149SequenceListing.txt, created on May 5, 2022, comprising 19,241 bytes, submitted concurrently with the filing of this application is incorporated herein by reference.

### FIELD AND BACKGROUND OF THE INVENTION

The present invention, in some embodiments thereof, relates to self-activating fusion proteins which comprise a light generating moiety and a light transducing moiety capable of luminescent signaling.

In recent years, there has been a growing interest in the field of synthetic cells for therapeutics, diagnostics and research on the origin of life. These cell-mimicking microparticles are designed from the bottom-up to reconstitute various processes of living cells or perform new functionalities that do not exist in nature. Protein expression, ATP production, DNA replication and cytoskeleton re-arrangement were successfully reconstructed in synthetic cells, yielding new insights in isolated and controlled environments. Recently, an increasing number of studies are focusing on potential clinical uses of these cells for diagnostics and therapeutics. Synthetic beta cells and therapeutic protein-producing synthetic cells are two examples of artificial constructs that have been tested in animal models in-vivo. Yet, to efficiently implement synthetic cell technologies in clinical settings, integration of multiple cellular abilities into a minimal functioning cell is required.

Of particular interest for utilizing synthetic cells in living tissue is cell signaling. Previous studies demonstrated the construction of chemical communication pathways in synthetic cells for coupling with bacterial cells and within synthetic cells. For example, isopropyl b-D-1-thiogalactopyranoside (IPTG), arabinose and C6-HSL (N-(3-oxohexanoyl)-L-homoserine lactone) have been used as signaling molecules to control gene expression and differentiation in synthetic cells. Light has also been used to stimulate cellular processes in synthetic cells. UV radiation was used to unlock DNA photo-caging for initiation of transcription, and blue light was applied to induce cellular adhesion of synthetic cells to a substrate using light-activated dimerization of the iLID (improved light-induced dimer) and sspB-Micro proteins. Blue light triggered ATP production in synthetic cells was demonstrated by coupling the proton gradient generated by bacteriorhodopsin to drive ATP synthase activity. Nevertheless, in order to use light in the visible spectrum to activate synthetic cells or natural light-responsive cells in vivo, invasive transplantations of external light sources are required. An alternative to using external light sources is to generate light in the synthetic cell themselves. This can be achieved by exploiting bioluminescent reactions catalyzed by enzymes from the luciferase family, which have been widely used in research for biological reporter assays and in-vivo imaging.

Background art includes Kim et al. Elife 8 (2019): e43826.; Li, Ting, et al. Nature communications 12.1 (2021): 1-10; Proshkina, G. M., et al. Journal of Photochemistry and Photobiology B: Biology 188 (2018): 107-115; Bartelt, Solveig Mareike, et al. Chemical Communications 54.8 (2018): 948-951; Chakraborty, Taniya, et al. Chemical Communications 55.64 (2019): 9448-9451; International Application No. WO2011005978A9, US Patent Application No. 20180044397A1.

### SUMMARY OF THE INVENTION

According to an aspect of the present invention there is provided a composition of matter comprising a fusion protein attached to the outer surface of a substrate, wherein the fusion protein comprises a light-generating protein and a light-transducing protein.

According to an aspect of the present invention there is provided a lipidated fusion protein comprising a light-generating protein and a light-transducing protein.

According to an aspect of the present invention there is provided a method of isolating an analyte comprising:

(a) contacting a solution which comprises the analyte with the composition of matter described herein, wherein the contacting is effected in the presence of a substrate that is capable of inducing emission of a photon by the light-generating protein; wherein the emission of the photon by the light generating protein is capable of inducing a response in the light-transducing protein that increases binding thereof to the target agent; and

(b) removing non-bound components present in the solution, thereby isolatinghe analyte.

According to an aspect of the present invention there is provided a method of targeting the composition of matter described herein to a particle having an outer surface, wherein a binding moiety is attached to the outer surface of the particle, the binding moiety being capable of binding to the light transducing protein upon emission of photons by the light generating protein, the method comprising contacting the composition of matter described herein with a substrate that is capable of inducing emission of a photon by the light-generating protein under conditions which promote binding of the composition of matter to the particle, thereby targeting the composition of matter described herein to the particle.

According to an aspect of the present invention there is provided a topical composition comprising a membrane of lipids which reduces penetration of ultraviolet light and allows penetration of visible light.

According to embodiments of the present invention, the light-generating protein comprises an inducible light generating protein.

According to embodiments of the present invention, the inducible light generating protein is a luciferase.

According to embodiments of the present invention, the light-generating protein is a photoprotein.

According to embodiments of the present invention, the photoprotein is Aequorin.

According to embodiments of the present invention, the luciferase comprises Gaussia luciferase or Renilla luciferase.

According to embodiments of the present invention, the light transducing protein is a light-gated ion channel.

According to embodiments of the present invention, the light transducing protein is improved light-induced dimer (iLID).

According to embodiments of the present invention, the light-generating protein is attached to the light-transducing protein via a linker peptide.

According to embodiments of the present invention, the linker peptide is at least 5 amino acids.

According to embodiments of the present invention, the fusion protein is lipidated.

According to embodiments of the present invention, the lipid of the lipidated fusion protein is attached to the light generating protein.

According to embodiments of the present invention, the fusion protein further comprises a cell membrane targeting moiety.

According to embodiments of the present invention, the cell comprises a synthetic cell.

According to embodiments of the present invention, the substrate comprises a lipid particle.

According to embodiments of the present invention, the lipid particle comprises a nickel chelating lipid and the light generating protein comprises a histidine tag.

According to embodiments of the present invention, the substrate comprises a bead or a resin.

According to embodiments of the present invention, the removing comprises separting the substrate from the solution.

According to embodiments of the present invention, the solution a biological

According to embodiments of the present invention, the biological fluid is selected from the group consisting of whole blood, serum, urine, cerebrospinal fluid, semen and saliva.

According to embodiments of the present invention, the substrate comprises a bead or a resin.

According to embodiments of the present invention, the light protein is iLID.

According to embodiments of the present invention, the substrate is a synthetic particle.

According to embodiments of the present invention, the particle having an outer surface is a biological cell.

According to embodiments of the present invention, the particle having an outer surface is a synthetic particle.

According to embodiments of the present invention, the synthetic particle is a nanoparticle.

According to embodiments of the present invention, the light-transducing protein is iLID.

According to embodiments of the present invention, the synthetic particle comprises a therapeutic or diagnostic agent.

According to embodiments of the present invention, the targeting is effected in vivo.

According to embodiments of the present invention, the targeting is effected ex vivo.

According to embodiments of the present invention, the light generating protein comprises a luciferase.

According to embodiments of the present invention, the substrate comprises a luciferin.

According to embodiments of the present invention, the luciferin is Coelenterazine (CTZ) or derivative thereof.

According to embodiments of the present invention, the lipids comprise phospholipids.

According to embodiments of the present invention, the lipids have a chain length of at least 16 carbons and wherein at least one of said 16 carbons is unsaturated.

According to embodiments of the present invention, the membrane comprises a lipid bilayer.

According to embodiments of the present invention, the lipids of said lipid bilayer have a chain length of at least 12 carbons.

According to embodiments of the present invention, the membrane comprises a lipid monolayer.

According to embodiments of the present invention, the lipids are comprised in a particle.

According to embodiments of the present invention, the composition is formulated as eye drops.

According to embodiments of the present invention, the composition a

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

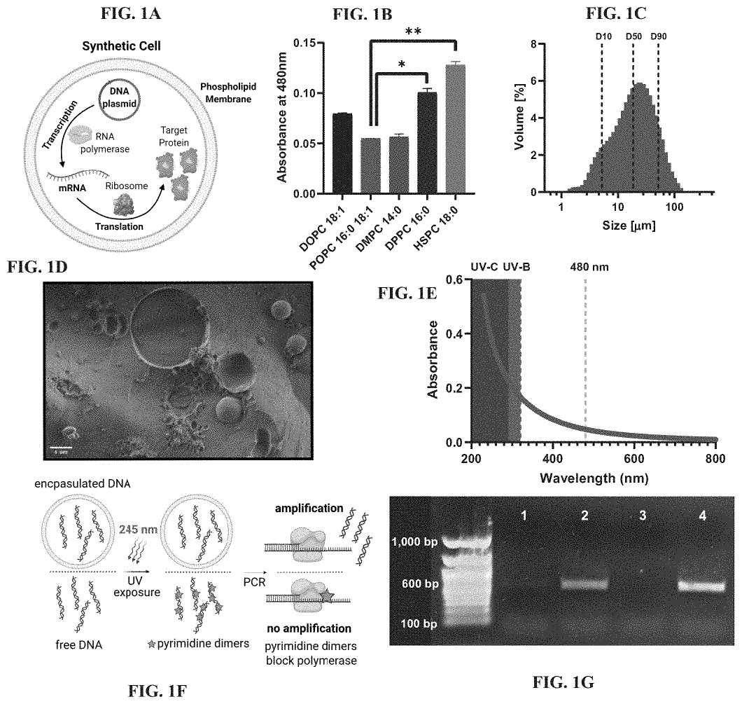

FIGS. 1A-G. Optimizing the lipid composition for light-interacting synthetic cells. A, Illustration of a protein producing synthetic cell. B, The effect of the lipid membrane composition on blue light absorbance in liposomes. Data is expressed as the mean±standard deviation (n=2 independent samples). Nested one-way ANOVA with multiple comparisons test adjusted P values; *P=0.0475, **P<0.0053. C, Size distribution of POPC:cholesterol synthetic cells measured with light scattering. Data is expressed as mean of n=3 independent samples. D, Morphology of POPC:cholesterol synthetic cells imaged with cryoSEM. E, The Absorbance spectrum of POPC phospholipid membrane in liposomes. F, A linear 600-bp DNA oligonucleotide encapsulated in a synthetic cell or free in solution was exposed to UV radiation. Resulting formation of pyrimidine dimers was detected using PCR amplification. G, Gel electrophoresis of the PCR product of linear DNA after exposure to UV radiation with and without encapsulation in synthetic cells. Lane 1: free DNA exposed to UV. Lane 2: encapsulated DNA exposed to UV. Lane 3: no DNA control. Lane 4: untreated DNA control.

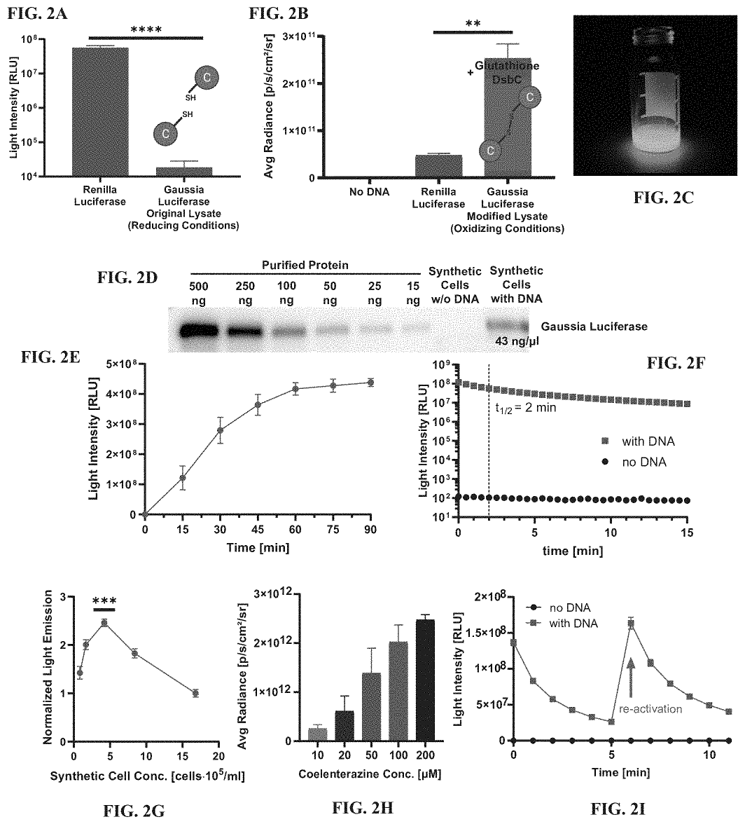

FIGS. 2A-I. Engineering light-generating synthetic cells. A, Comparison of light emission in cell-free protein synthesis reactions expressing Renilla Luciferase (Rluc) or Gaussia luciferase (Gluc) using unmodified lysate (reducing conditions). Emission measured in relative light unites (RLU) with TECAN plate reader spectrophotometer (n=3 independent samples). Nested T test adjusted P value; ****P<0.0001. B, Light emission in synthetic cells expressing Gluc using modified lysate with addition of glutathione and disulfide bond isomerase C (DsbC) to produce an oxidizing environment in the synthetic cells, compared to light emission in Rluc-expressing synthetic cells. Emission was measured in average radiance using IVIS spectrum CT (sr=steradian). Nested one-way ANOVA with multiple comparisons test adjusted P value; **P=0.0047 (n=2 independent samples). C, Light emission from a Gluc-expressing synthetic cell solution. D, Western blot quantification of Gluc production in synthetic cells. E, Gluc production kinetics in synthetic cells at 37° C. (n=3 independent samples). F, Kinetics of the Gluc enzymatic reaction in synthetic cells after one addition of coelenterazine (n=3 independent samples). G, Light emission from Gluc-expressing synthetic cells diluted to different concentrations after incubation. Nested one-way ANOVA with multiple comparisons test adjusted P value; ***P<0.0006 (n=3 independent samples). H, Light emission under a range of coelenterazine concentrations in Gluc-expressing synthetic cells (between n=2 and n=3 independent samples) I, Temporal control over light emission in Gluc-expressing synthetic cells with two coelenterazine additions (second addition marked with an arrow, n=3 independent samples) (i). Data is expressed as a mean±standard deviation.

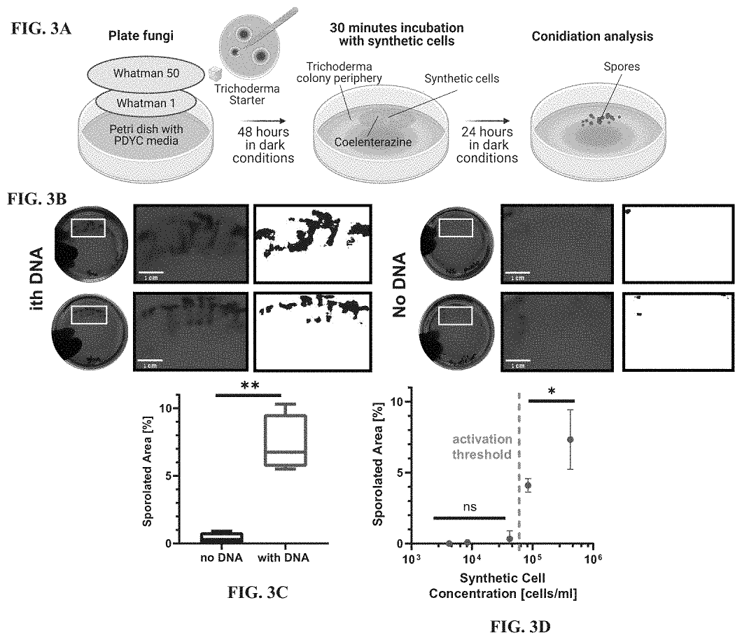

FIGS. 3A-D. Activation of fungal cells using light-producing synthetic cells. A, Illustration of the experimental procedure for photo-activation of conidiation in Tricoderma atroviride with Gluc-expressing synthetic cells. B, Representative images of Tricoderma atroviride plates after exposure to Gluc-expressing synthetic cells or Synthetic cells without DNA. To the right of each plate image are a zoom in image and a black and white thresholded image of the plate area marked with a white rectangle in which the synthetic cells were localized. C, Quantitative analysis of the sporulated area out of the total area exposed to synthetic cells. Data is expressed as min-to-max box plot±standard deviation (between n=4 to n=5 independent samples). Welch's T test P value; **P=0.0085. D, The effect of varying synthetic cell concentration on photo-activation of conidiation in Tricoderma atroviride. Data is expressed as a mean±standard deviation. (between n=3 to n=4 independent samples). One-way ANOVA with multiple comparisons test adjusted P value; *P<0.0313.

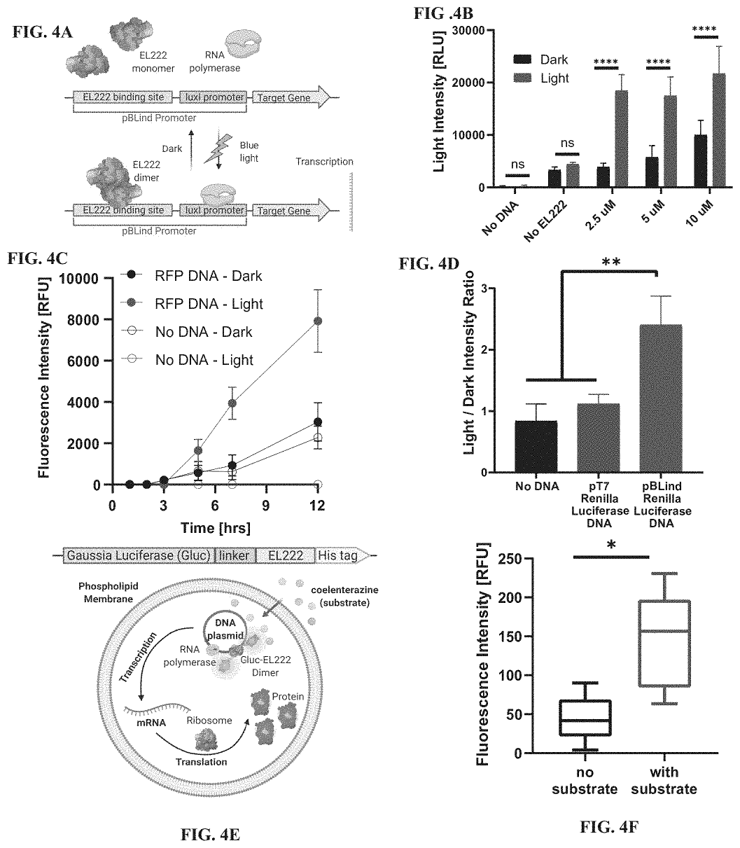

FIGS. 4A-F Bioluminescent signaling activates transcription in synthetic cells. A, Illustration of the light-dependent transcription mechanism mediated by the transcription factor EL222. B, EL222 concentration affected the production of Renilla luciferase (Rluc) in cell-free protein synthesis (CFPS) reactions under light and dark conditions. Data is expressed as a mean±standard deviation (between n=5 to n=6 independent samples). Two-way ANOVA with multiple comparisons test adjusted P value; ****P<0.0001. C, RFP production in CFPS reactions supplemented with EL222 under dark and light conditions, with or without RFP DNA. Data is expressed as a mean±s.e.m. (between n=4 to n=6 independent samples). D, Light-to-dark ratio of Rluc expression in synthetic cells containing EL222 and a DNA plasmid expressing Rluc under different promoters. Data is expressed as a mean±standard deviation (between n=3 to n=4 independent samples). One-way ANOVA with multiple comparisons test adjusted P value; **P<0.005. E, A block diagram of the Gluc-EL222 fusion protein elements. Below, a schematic representation of a synthetic cells containing the Gluc-EL222 fusion protein for bioluminescent activation of transcription. F, RFP production in synthetic cells containing the Gluc-EL222 protein with or without addition of coelenterazine. Data is expressed as min-to-max box plot±standard deviation (n=5 independent samples). T test P value; *P=0.0134.

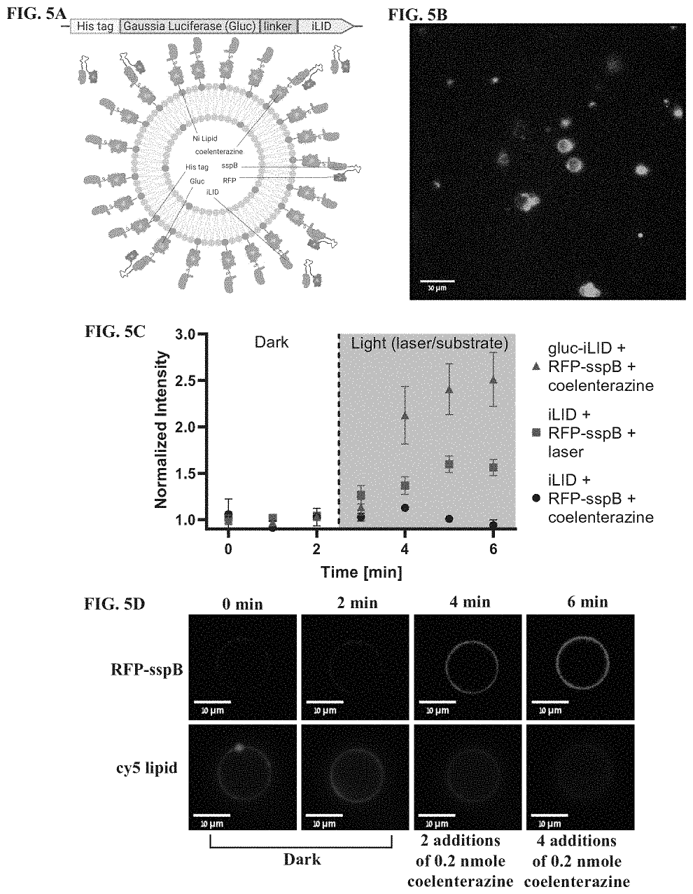

FIGS. 5A-D. Bioluminescence-activated membrane recuritment in synthetic cells. A, A block diagram of the Gaussia luciferase (Gluc)-iLID fusion protein elements. Below, a schematic representation of protein recruitment to the synthetic cell membrane by hetero-dimerization of the fusion protein Gluc-iLID and RFP-sspB Nano. B,

Microscopy image of luciferase light emission from synthetic cells with membrane-bound Gluc-EL222 after coelenterazine addition. C, Membrane recruitment of RFP conjugated to sspB-Nano with iLID or Gluc-iLID using 488 nm laser illumination or by addition of coelenterazine to activate the bioluminescent reaction. RFP intensity is normalized to the average intensity of each sample in the dark conditions. Data is expressed as a mean±s.e.m. (n=3 for iLID+coelenterazine, n=10 for iLID+laser, n=14 for gluc-iLID+coelenterazine). D, Single-cell images of RFP-sspB Nano recruitment to a synthetic cell with membrane-bound Gluc-iLID in the dark and after two and four doses of 0.2 nmol coelenterazine. Bottom channel displayes the cell's DOPE-Cy5 fluorescent lipid that composes 0.5% of the synthetic cell's membrane.

### DESCRIPTION OF SPECIFIC EMBODIMENTS OF THE INVENTION

The present invention, in some embodiments thereof, relates to self-activating fusion proteins which comprise a light generating moiety and a light transducing moiety capable of luminescent signaling.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Development of regulated cellular processes and signaling methods in synthetic cells is essential for their integration with living materials. Light is an attractive tool to achieve this, but the limited penetration depth into tissue of visible light restricts its usability for in-vivo applications. The present inventors have now designed and implemented bioluminescent intercellular and intracellular signaling mechanisms in synthetic cells, dismissing the need for an external light source.

First, light generating synthetic cells (SCs) were engineered with an optimized lipid membrane (FIGS. 1A-E) and internal composition (FIGS. 2A-I), to maximize luciferase expression levels and enable high-intensity emission. The SCs were composed of giant unilamellar vesicles (GUVs) encapsulating a bacterial-based cell-free protein synthesis (CFPS) system.

Next, intracellular bioluminescent signaling processes were engineered in SCs. In order to utilize light-responsive proteins that required higher intensities, self-activating fusion proteins were engineered by coupling Gaussia luciferase (Gluc) with photo-responsive proteins, facilitating their activation by bioluminescence resonance energy transfer (BRET). This approach was used to control transcription in SCs using a bioluminescent fusion protein of Gluc and the light-activated transcription factor EL222 (FIGS. 4A-F). Light-controlled activation of membrane recruitment in SCs was achieved as well, with an additional BRET-based signaling mechanism utilizing a fusion protein of Gluc and iLID, that dimerized with a sspB-tagged protein when the bioluminescent reaction was initiated (FIGS. 5A-D). Altogether, these SC signaling functionalities present opportunities for bioluminescent activation and control of synthetic and natural cells alike.

Thus, according to a first aspect of the present invention, there is provided a composition of matter comprising a fusion protein attached to an outer surface of a substrate, wherein the fusion protein comprises a first amino acid sequence and a second amino acid sequence, wherein the first amino acid sequence comprises a light-generating protein, and the second amino acid sequence comprises a light-transducing protein.

As used herein, the term “fusion polypeptide” or “fusion protein” refers to a protein created by joining two or more polypeptide sequences together. In one embodiment, the “fusion protein” is a recombinant protein of two or more proteins which are joined by a peptide bond.

Light generating proteins are luminescent proteins that are capable of converting chemical energy into electromagnetic radiation through an oxidation reaction.

According to a particular embodiment, the light generating protein is inducible and only emits light in the presence of an inducer (e.g. substrate molecule).

According to a particular embodiment, the light generating protein is inducible and only emits light in the absence of a repressor (e.g. substrate molecule).

In one embodiment, the light generating protein is a functional luciferase. The luciferase can be derived from any source, such as those from Gaussia, Renilla reniformis, and firefly, and include derivatives that are able to emit a photon of light in response to contact with a substrate molecule. Substrates include, but are not limited to, luciferin, coelenterazine and other modified substrates. The substrate is matched with the light generating protein. For Example, firefly luciferase uses luciferin as a substrate while Renilla luciferase uses coelenterazine as a substrate. Those skilled in the art will appreciate that substrates may be chosen to affect the kinetics, membrane permeability, turn over or signal strength of the emission. Luciferases oxidize luciferin to produce oxyluciferin and light energy. The chemical reaction can occur intracellularly, extracellularly, or be membrane anchored.

In another embodiment, the light generating protein is a photoprotein (e.g., Aequorin and Obelin). A photoprotein coordinates to a luciferin and to molecular oxygen, and the oxidation reaction is then triggered by a stimulus (e.g., by Ca²⁺ ions).

The light generating protein can be, e.g., wild-type or mutant, e.g., modified to enhance luminescence or modified to enhance the light emission wavelength or the light absorbance wavelength. Non-limiting examples of mutant luminescent proteins include GLuc M431, GLuc Y97W, GLuc 190L, Monsta (GLuc having F89W, 190L, H95E, and Y97W mutations), and GLuc4 (L30S, L40P, M43V).

According to a particular embodiment, the light-generating protein emits blue light—e.g. Gaussia Luciferase (GLuc). The light-generating protein may have an amino acid sequence at least 90 identical, at least 91% identical, at least 92% identical, at least 93% identical, at least 94% identical, at least 95% identical, at least 96% identical, at least 97% identical at least 98% identical, or at least 99% identical to SEQ ID NO: 2, which is able to generate blue light.

Percent identity can be determined using any homology comparison software, including for example, the BlastP software of the National Center of Biotechnology Information (NCBI) such as by using default parameters.

Other exemplary sequence alignment programs that may be used to determine % homology or identity between two sequences include, but are not limited to, the FASTA package (including rigorous (SSEARCH, LALIGN, GGSEARCH and GLSEARCH) and heuristic (FASTA, FASTX/Y, TFASTX/Y and FASTS/M/F) algorithms, the EMBOSS package (Needle, stretcher, water and matcher), the BLAST programs (including, but not limited to BLASTN, BLASTX, TBLASTX, BLASTP, TBLASTN), megablast and BLAT. In some embodiments, the sequence alignment program is BLASTN. For example, 95% homology refers to 95% sequence identity determined by BLASTN, by combining all non-overlapping alignment segments (BLAST HSPs), summing their numbers of identical matches and dividing this sum with the length of the shorter sequence.

In some embodiments, the sequence alignment program is a basic local alignment program, e.g., BLAST. In some embodiments, the sequence alignment program is a pairwise global alignment program. In some embodiments, the pairwise global alignment program is used for protein-protein alignments. In some embodiments, the pairwise global alignment program is Needle. In some embodiments, the sequence alignment program is a multiple alignment program. In some embodiments, the multiple alignment program is MAFFT. In some embodiments, the sequence alignment program is a whole genome alignment program. In some embodiments, the whole genome alignment is performed using BLASTN. In some embodiments, BLASTN is utilized without any changes to the default parameters.

The term “light transducing protein” refers to a protein that can covert light energy (i.e., photons) into an effector function in a single component system (e.g., opsins from microbes) or in a more complex multi component signaling cascade, (e.g., G protein-coupled receptors) and signaling pathways (e.g. human rhodopsin).

According to a particular embodiment, the light transducing protein is a flavin □ based photo-responsive proteins (such as iLID and EL222).

The light transducing protein may be at least 90% identical, at least 91% identical, at least 92% identical, at least 93% identical, at least 94% identical, at least 95% identical, at least 96% identical, at least 97% identical at least 98% identical, or at least 99% identical to SEQ ID NO: 2 or SEQ ID NO: 4.

According to other embodiments, the light transducing is a light-gated ion channel such as a channelrhodopsin.

The term “channelrhodopsin” as used herein, relates to the subfamily of opsin proteins that function as light-gated ion channels

Another non-limiting example of a class of light-transducing protein includes the opsins, for example Chlamydomonas channelrhodopsin-2 (ChR2), or Volvox channelrhodopsin-1 (VChR1). Natronomonas halorhodopsin (NpHR), opsin from Acetabularia acetabulum (AR) bacteriorhodopsin from H. salinarum (BR), Guillardia theta rhodopsin-3 (GtR3), as well as opsins from other diverse hosts including such non-limiting examples as Cryptomonas, Guillardia, Mesostigma, Dunaliella, Gloeobacter, and the like. In some embodiments described herein, the light-transducing protein comprises Chlamydomonas channelrhodopsi 2 (ChR2), or Volvox channelrhodopsin-1 (VChR,1).

Different light generating proteins have different emission spectra. For example, Gaussia Luciferase (GLuc) emits blue light with a wavelength at 477nm, Other known luciferases emit green, red, infrared. or yellow light after contact with the appropriate substrate. Thus a light-generating protein can be matched with the light-transducing protein to result in maximal efficiency of signal generation after addition of the substrate. in addition to emission spectra of the light-generating proteins the kinetics of activation and decay of the light.- generating protein must he considered. Some light-generating proteins have flash kinetics which include rapid decay, while others with longer decay times may be advantageous for other applications.

Linking of the light-generating protein to the light-transducing protein may be effected using any method known in the art provided that the linking does not substantially interfere with the bioactivity of the individual proteins.

The light-generating proteins may be linked to the light-transducing proteins through a linking moiety. In one embodiment, the N-terminus of the light-generating protein is attached to the C-terminus of the light-transducing protein. In another embodiment, the N-terminus of the light-transducing protein is attached to the C-terminus of the light-generating protein.

Examples of linking moieties include, but are not limited to, a simple covalent bond, a flexible peptide linker, a disulfide bridge or a polymer such as polyethylene glycol (PEG). Peptide linkers may be entirely artificial (e.g., comprising at least 5, at least 10, at least 20 or any number between 2 to 50 amino acid residues independently selected from the group consisting of glycine, serine, asparagine, threonine, proline, valine and alanine) including their natural posttranslational modification e.g. O- and N-glycosylations or adopted from naturally occurring proteins. Disulfide bridge formation can be achieved, e.g., by addition of cysteine residues, as further described herein below. Linking through polyethylene glycols (PEG) can be achieved by reaction of monomers having free cysteines with multifunctional PEGs, such as linear bis-maleimide PEGs. Alternatively, linking can be performed though the glycans on the monomer after their oxidation to aldehyde form and using multifunctional PEGs containing aldehyde-reactive groups.

Selection of the position of the link between the two monomers should take into account that the link should not substantially interfere with the ability of the fusion protein to generate light and transduce the light into a function.

Thus, according to one embodiment the linker comprises the amino acid sequence as set forth in SEQ ID NO: 8.

Non-peptide linkers are also possible. For example, alkyl linkers such as —NH— —(CH₂)ₛ—C(O)—, wherein s=2-20 could be used. These alkyl linkers may further be substituted by any non-sterically hindering group such as lower alkyl (e.g., C₁-C₆) lower acyl, halogen (e.g., Cl, Br), CN, NH_{2,} phenyl, etc. An exemplary non-peptide linker is a PEG linker.

According to another embodiment the link is effected using a coupling agent.

The term “coupling agent”, as used herein, refers to a reagent that can catalyze or form a bond between two or more functional groups intra-molecularly, inter-molecularly or both. Coupling agents are widely used to increase polymeric networks and promote crosslinking between polymeric chains, hence, in the context of some embodiments of the present invention, the coupling agent is such that can promote crosslinking between polymeric chains; or such that can promote crosslinking between amino functional groups and carboxylic functional groups, or between other chemically compatible functional groups of polymeric chains. In some embodiments of the present invention the term “coupling agent” may be replaced with the term “crosslinking agent”. In some embodiments, one of the polymers serves as the coupling agent and acts as a crosslinking polymer.

By “chemically compatible” it is meant that two or more types of functional groups can react with one another so as to form a bond.

Exemplary functional groups which are typically present in gelatins and alginates include, but are not limited to, amines (mostly primary amines —NH₂), carboxyls (—CO₂H), sulfhydryls and hydroxyls (—SH and —OH respectively), and carbonyls (—COH aldehydes and —CO— ketones).

Primary amines occur at the N-terminus of polypeptide chains (called the alpha-amine), at the side chain of lysine (Lys, K) residues (the epsilon-amine), as found in gelatin, as well as in various naturally occurring polysaccharides and aminoglycosides. Because of its positive charge at physiologic conditions, primary amines are usually outward-facing (i.e., found on the outer surface) of proteins and other macromolecules; thus, they are usually accessible for conjugation.

Carboxyls occur at the C-terminus of polypeptide chain, at the side chains of aspartic acid (Asp, D) and glutamic acid (Glu, E), as well as in naturally occurring aminoglycosides and polysaccharides such as alginate. Like primary amines, carboxyls are usually on the surface of large polymeric compounds such as proteins and polysaccharides.

Sulfhydryls and hydroxyls occur in the side chain of cysteine (Cys, C) and serine, (Ser, S) respectively. Hydroxyls are abundant in polysaccharides and aminoglycosides.

Carbonyls as ketones or aldehydes can be form in glycoproteins, glycosides and polysaccharides by various oxidizing processes, synthetic and/or natural.

According to some embodiments of the present invention, the coupling agent can be selected according to the type of functional groups and the nature of the crosslinking bond that can be formed therebetween. For example, carboxyl coupling directly to an amine can be afforded using a carbodiimide type coupling agent, such as EDC; amines may be coupled to carboxyls, carbonyls and other reactive functional groups by N-hydroxysuccinimide esters (NHS-esters), imidoester, PFP-ester or hydroxymethyl phosphine; sulfhydryls may be coupled to carboxyls, carbonyls, amines and other reactive functional groups by maleimide, haloacetyl (bromo- or iodo-), pyridyldisulfide and vinyl sulfone; aldehydes as in oxidized carbohydrates, may be coupled to other reactive functional groups with hydrazide; and hydroxyl may be coupled to carboxyls, carbonyls, amines and other reactive functional groups with isocyanate.

Hence, suitable coupling agents that can be used in some embodiments of the present invention include, but are not limited to, carbodiimides, NHS-esters, imidoesters, PFP-esters or hydroxymethyl phosphines.

The proteins of the present invention can be generated using recombinant techniques such as described by Bitter et al. (1987) Methods in Enzymol. 153:516-544; Studier et al. (1990) Methods in Enzymol. 185:60-89; Brisson et al. (1984) Nature 310:511-514; Takamatsu et al. (1987) EMBO J. 6:307-311; Coruzzi et al. (1984) EMBO J. 3:1671-1680; Brogli et al. (1984) Science 224:838-843; Gurley et al. (1986) Mol. Cell. Biol. 6:559-565 and Weissbach & Weissbach, 1988, Methods for Plant Molecular Biology, Academic Press, NY, Section VIII, pp 421-463. It will be appreciated that when the proteins are linked to one another via a peptide linker, the fusion protein itself may be generated using recombinant techniques. When the proteins are linked to one another via a non-peptide linker, the light generating protein and the light-transducing protein may be generated by recombinant technology and subsequently the two proteins may be linked as described herein above.

For example, a nucleic acid sequence (e.g. SEQ ID NO: 1) encoding a light-generating protein of the present invention is ligated to a nucleic acid sequence (e.g. SEQ ID NO: 3 or 5) which includes an in-frame sequence encoding a light- transducing protein.

Exemplary nucleic acid sequences which may be used to express the fusion proteins are set forth in SEQ ID NOs: 9 or 11.

Exemplary amino acid sequences of the fusion proteins according to embodiments of the present invention are set forth in SEQ ID NOs: 10 or 12.

Also provided is an expression vector, comprising the isolated polynucleotide of some embodiments of the invention. According to one embodiment, the polynucleotide sequence is operably linked to a cis- acting regulatory element.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The nucleic acid construct of some embodiments of the invention typically includes a signal sequence for secretion from a host cell in which it is placed. In one embodiment, the signal sequence for this purpose is a mammalian signal sequence.

Preferably, the promoter utilized by the nucleic acid construct of some embodiments of the invention is active in the specific cell population transformed.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Also provided are cells which comprise the polynucleotides/expression vectors as described herein.

Suitable host cells for cloning or expression include prokaryotic or eukaryotic cells. See e.g. Charlton, Methods in Molecular Biology, Vol. 248 (B. K. C. Lo, ed., Humana Press, Totowa, N. J., 2003), pp. 245-254, and describing expression of antibody fragments in E. coli; see Gerngross, Nat. Biotech. 22:1409-1414 (2004), and Li et al., Nat. Biotech. 24:210-215 (2006) for suitable fungi and yeast strains; and see e.g., U.S. Pat. Nos. 5,959,177, 6,040,498, 6,420,548, 7,125,978, and 6,417,429 for suitable plant cell cultures which can also be utilized as hosts.

After expression, the fusion protein (or fusion protein components) may be isolated from the cells in a soluble fraction and can be further purified.

Recovery of the fusion protein (or fusion protein components) may be effected following an appropriate time in culture. The phrase “recovering the recombinant polypeptide or fusion protein” refers to collecting the whole fermentation medium containing the polypeptide or fusion protein and need not imply additional steps of separation or purification.

Notwithstanding the above, proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Molecules of the present invention are preferably retrieved in “substantially pure” form. As used herein, “substantially pure” refers to a purity that allows for the effective use of the protein in the applications, described herein.

The present invention contemplates that the fusion protein is comprised inside a particle e.g. a biological cell or a synthetic cell having a lipid (e.g. phospholipid) membrane.

Exemplary particles that may be used according to this aspect of the present invention include, but are not limited to polymeric particles, microcapsules, liposomes, microspheres, microemulsions, nanoparticles, nanocapsules, nano-spheres, nano-liposomes, nano-emulsions and nanotubes.

In one embodiment, the particle is a biological particle—e.g. an erythrocyte or a cell ghost.

In another embodiment, the particle is a non-biological particle—i.e. not a cell. According to a particular embodiment, the particles are microparticles (e.g. giant unilamellar vesicle).

The particles may be of any shape, including, without limitation, elongated particle shapes, such as nanowires, or irregular shapes, in addition to more regular shapes, such as generally spherical, hexagonal and cubic nanoparticles. According to one embodiment, the particles are generally spherical.

The particles of this aspect of the present invention may have a charged surface (i.e., positively charged or negatively charged) or a neutral surface.

Agents which are used to fabricate the particles may be selected according to the desired charge required on the outer surface of the particles.

Thus, for example if a negatively charged surface is desired, the particles may be fabricated from negatively charged lipids (i.e. anionic phospholipids) such as described herein below.

When a positively charged surface is desired, the particles may be fabricated from positively charged lipids (i.e. cationic phospholipids), such as described herein below.

As mentioned, non-charged particles are also contemplated by the present invention. Such particles may be fabricated from neutral lipids such as phosphatidylethanolamine or dioleilphosphatidylethanolamine (DOPE).

It will be appreciated that combinations of different lipids may be used to fabricate the particles of the present invention, including a mixture of more than one cationic lipid, a mixture of more than one anionic lipid, a mixture of more than one neutral lipid, a mixture of at least one cationic lipid and at least one anionic lipid, a mixture of at least one cationic lipid and at least one neutral lipid, a mixture of at least one anionic lipid and at least one neutral lipid and additional combinations of the above.

In addition, ionizable lipids may be used. In addition, polymer-lipid based formulations may be used.

There are numerous polymers which may be attached to lipids. Polymers typically used as lipid modifiers include, without being limited thereto: polyethylene glycol (PEG), polysialic acid, polylactic (also termed polylactide), polyglycolic acid (also termed polyglycolide), apolylactie-polyglycolic acid' polyvinyl alcohol, polyvinylpyrrolidone, polymethoxazoline, polyethyloxazoline, polyllydroxyetlyloxazolille, solyhydroxypryloxazoline, polyaspartarllide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose.

The polymers may be employed as homopolymers or as block or random copolymers.

The particles may also include other components. Examples of such other components includes, without being limited thereto, fatty alcohols, fatty acids, and/or cholesterol esters or any other pharmaceutically acceptable excipients which may affect the surface charge, the membrane fluidity and assist in the incorporation of the biologically active lipid into the lipid assembly. Examples of sterols include cholesterol, cholesterol hemisuccinate, cholesterol sulfate, or any other derivatives of cholesterol. Preferred lipid assemblies according the invention include either those which form a micelle (typically when the assembly is absent from a lipid matrix) or those which form a liposome (typically, when a lipid matrix is present).

In one embodiment, the particle is a lipid-based microparticle. The core of the particle may be hydrophilic or hydrophobic. The core of the lipid-based microparticle may comprise some lipids, such that it is not fully hydrophilic.

In a specific embodiment, the particle is a liposome. As used herein and as recognized in the art, liposomes include any synthetic (i.e., not naturally occurring) structure composed of lipid bilayers, which enclose a volume. Liposomes include emulsions, foams, micelles, insoluble monolayers, liquid crystals, phospholipid dispersions, lamellar layers and the like. The liposomes may be prepared by any of the known methods in the art [Monkkonen, J. et al., 1994, J. Drug Target, 2:299-308; Monkkonen, J. et al., 1993, Calcif. Tissue Int., 53:139-145; Lasic D D., Liposomes Technology Inc., Elsevier, 1993, 63-105. (chapter 3); Winterhalter M, Lasic D D, Chem Phys Lipids, 1993 September; 64(1-3):35-43].

The liposomes may be unilamellar or may be multilamellar. Unilamellar liposomes may be preferred in some instances as they represent a larger surface area per lipid mass. Suitable liposomes in accordance with the invention are preferably non-toxic. The liposomes may be fabricated from a single phospholipid or mixtures of phospholipids. The liposomes may also comprise other lipid materials such as cholesterol. For fabricating liposomes with a negative electrical surface potential, acidic phospho- or sphingo- or other synthetic-lipids may be used. Preferably, the lipids have a high partition coefficient into lipid bilayers and a low desorption rate from the lipid assembly. Exemplary phospholipids that may be used for fabricating liposomes with a negative electrical surface potential include, but are not limited to phosphatidylserine, phosphatidic acid, phosphatidylcholine and phosphatidyl glycerol.

Other negatively charged lipids which are not liposome forming lipids that may be used are sphingolipids such as cerebroside sulfate, and various gangliosides.

The most commonly used and commercially available lipids derivatized into lipopolymers are those based on phosphatidyl ethanolamine (PE), usually distearylphosphatidylethanolamine (DSPE).

The lipid phase of the liposome may comprise a physiologically acceptable liposome forming lipid or a combination of physiologically acceptable liposome forming lipids for medical or veterinarian applications. Liposome-forming lipids are typically those having a glycerol backbone wherein at least one of the hydrofoil groups is substituted with an acyl chain, a phosphate group, a combination or derivatives of same and may contain a chemically reactive group (such as an as amine imine, acids ester, aldelhyde or alcohol) at the headgroup. Typically, the acyl chain is between 12 to about 24 carbon atoms in length, and has varying degrees of saturation being fully, partially or non-hydrogenated lipids. Further, the lipid matrix may be of natural source, semi-synthetic or fully synthetic lipid, and neutral, negatively or positively charged.

According to one embodiment, the lipid phase comprises phospholipids.

The phospholipids may be a glycerophospholipid. Examples of glycerophospholipid include, without being limited thereto, phosphatidylglycerol (PG) including dimyristoyl phosphatidylglycerol (DMPG); phosphatidylcholine (PC), including egg yolk phosphatidylcholine and dimyristoyl phosphatidylcholine (DMPC), phosphatidic acid (PA), phosphatidylinositol (PI), phosphatidylserine (PS) and sphingomyelin (SM) and derivatives of the same.

Another group of lipid matrix employed according to the invention includes cationic lipids (monocationic or polycationic lipids). Cationic lipids typically consist of a lipophilic moiety, such as a sterol or the same glycerol backbone to which two acyl or two alkyl, or one acyl and one alkyl chain contribute the hydrophobic region of the amphipathic molecule, to form a lipid having an overall net positive charge.

Preferably, the head groups of the lipid carry the positive charge. Monocationic lipids may include, for example, 1,2-dimyristoyl-3-trimethylammonium propane (DMTAP) 1,2-dioleyloxy-3-(trimethylanino) propane (DOTAP), N-[-1-(2,3 -ditetradecyloxy)propyl]-N,N-dimethyl-N-hydroxyethylammonium bromide (DMRIE), N-[1(2,3 -dioleyloxy)propyl]-N,N-dimethyl-N-hydroxy ethyl-ammonium bromide (DORIE), N-[1-(2,3 -dioleyloxy) propyl];-N,N,N-trimethylammonium chloride (DOTMA); 3; N-(N′,N′-dimethylaminoethane) carbamoly]; cholesterol (DC-Chol), and I dimethyl-dioctadecylammonium (DDAB).

Examples of polycationic lipids include a similar lipoplilic moiety as with the mono cationic lipids, to which spermine or spermidine is attached. These include without being limited thereto, N- [2- [[2,5-bis[3-aminopropyl)amino]-1-oxopentyl]amino]ethyl]N,N dimethul-2,3 bis (1-oXo-9-octadecenyl) oXy];-1 propanaminium (DOSPA), and ceramide carbamoyl spermine (CCS).

The cationic lipids may be used alone, in combination with cholesterol, with neutral phospholipids or other known lipid assembly components. In addition, the cationic lipids may form part of a derivatized phospholipids such as the neutral lipid dioleoylphosphatidyl ethanolamine (DOPE) derivatized with polylysine to form a cationic lipopolymer.

According to a particular embodiment, the particle comprises a nickel-chelating lipid, as further described herein below.

The synthetic particles may comprise other components including, but not limited to cholesterol.

The present inventors have shown that the choice of phospholipid influences the amount of light transmission through the lipid membrane. Thus, selection of the particular lipid will depend on the choice of light generating protein used, the size of the particle and the application thereof. The present invention contemplates that the fusion protein may be attached to the outer surface of a substrate via a chemical or a physical bond. In one embodiment, the light transducing portion of the fusion protein is conjugated to the outer surface of the substrate. In another embodiment, the light generating portion of the fusion protein is conjugated to the outer surface of the substrate.

The substrate is typically a solid substrate such as a matrix, a bead e.g. magnetic bead), an agarose or a resin.

In another embodiment, the solid substrate may be a biological or synthetic cell, as described herein above. In the context of a biological cell or a synthetic cell, the fusion protein is attached to the exterior of the particle and is not exposed to the intracellular fluid.

Thus, the solid substrate may be a nanoparticle or a microparticle. The solid substrate may be a particle which comprises lipids in its exterior wall.

The exterior wall of the particle may comprise metal-chelating lipids comprising such moieties, such as 1,2-dioleoyl-sn-glycero-3-[(N-(5-amino-1-carboxypentyl)iminodiacetic acid)succinyl] (ammonium salt) (DOGS-NTA, e.g., DOGS -NTA-Ni), 1-paomitoyl-2-[8-[(E,E)-2′, 4′-hexadienolyloxyloctanoyl]-sn-glycero-3-N-[11-[N′,N′-bis[carboxymethyl]-3,6,9-trioxaundecanoyl] phosphatidylethanolamine (which chelates Cu through an IDA moiety), and lipid distearyl imino-diacetate (DSIDA, e.g., Cu-DSIDA).

In one embodiment, the fusion protein is attached to a lipid membrane targeting moiety.

Examples of lipid membrane targeting moieties include, but are not limited to lipids and cell membrane targeting peptides. In one embodiment, the lipid membrane targeting moiety is a moiety that interacts directly with a derivatized lipid of the membrane. Derivatized lipids include nickel-derivatized lipids (as described herein above); the targeting moiety may include a histidine tag.

Typically, lipid membrane targeting peptides have an amino acid composition containing either a high relative abundance of positively charged amino acids such as lysine or arginine, or have sequences that contain an alternating pattern of polar/charged amino acids and non-polar, hydrophobic amino acids.

By way of a non-limiting example, cell penetrating peptide (CPP) sequences may be used in order to enhance penetration of a phospholipid membrane. CPPs may include short and long versions of the protein transduction domain (PTD) of HIV TAT protein, such as for example, YARAAARQARA (SEQ ID NO: 13), YGRKKRR (SEQ ID NO: 14), YGRKKRRQRRR (SEQ ID NO: 15), or RRQRR (SEQ ID NO: 16)]. However, the disclosure is not so limited, and any suitable penetrating agent may be used, as known by those of skill in the art. In one embodiment the cell membrane targeting moiety 0 lipid) is attached to the light generating protein of the fusion protein. In another embodiment, the cell membrane targ g moiety (e.g. lipid) is attached to the light transducing protein of the fusion protein.

Thus, according to another aspect of the present invention there is provided a lipidated fusion protein comprising a light-generating protein and a light-transducing protein.

In one embodiment, the lipidated fusion protein is expressed in bacterial cells contain an N-terminal sequence which is derived from a leader peptide recognized by a bacterial lipidation system. After removal of the leader peptide during export through the inner membrane, the mature protein contains the N-terminal sequence of the leader peptide (e.g. CDQSSS-SEQ ID NO: 17) which is targeted by the lipidation system, resulting in lipid-acylation of the cysteine). It will be appreciated that in this embodiment, there is no linker sequence between the fusion protein itself and the lipidated portion of the protein—rather the fusion protein itself is directly lipidated.

In one embodiment, the signal sequence is part of an inner membrane bacterial (e.g. E. coli) lipoprotein.

One example of an inner membrane lipoprotein is NlpA. (new lipoprotein A). The first six amino acid of NipA can be used as an N terminal anchor (CDQSSS: SEQ ID NO: 17). Other examples of anchors that may find use with the invention include lipoproteins, Pullulanase of K. pneumoniae, which has the CDNSSS (SEQ ID NO: 18) mature lipoprotein. anchor, phage encoded celB, and E. coli acrE (envC).

In one embodiment, the lipid is covalently bound the fusion protein. In another embodiment, the lipid is non-covalently bound to the fusion protein. The lipid moieties could be a diacyl or triacyl lipid.

In one embodiment, the fusion protein undergoes fatty acylation (including modification of the N-terminal glycine of proteins by N-myristoylation and/or attachment of palmitate to internal cysteine residues). Protein prenylation involves attachment of farnesyl or geranylgeranyl moieties via thin-ether linkage to cysteine residues at or near the C-terminus. Attachment of each of these lipophilic groups is catalyzed by a distinct enzyme or set of enzymes: N-myristoyl transferase for N-myristoylation, palmitoyl acyl transferases for palmitoylation, and farnesyl or geranylgeranyl transferases for prenylation. The distinct nature of the lipid modification determines the strength of membrane interaction of the modified protein as well as the specificity of membrane targeting.

In one embodiment, the lipid is attached directly to the fusion protein. Alternatively, the lipid may be attached via a linker to the fusion protein.

The fusion proteins described herein have a myriad of uses. In one embodiment, the fusion proteins may be used to isolate analytes.

Thus, according to another aspect of the present invention there is provided a method of isolating an analyte comprising:

(a) contacting a solution which comprises the analyte with a fusion protein attached to the outer surface of a substrate (e.g. a bead or a resin), wherein the fusion protein comprises a light-generating protein and a light-transducing protein, wherein the contacting is effected in the presence of a substrate that is capable of inducing emission of a photon by the light-generating protein; wherein the emission of the photon by the light generating protein is capable of inducing a response in the light-transducing protein that increases binding thereof to said analyte; and

(b) removing non-bound components present in the solution, thereby isolating the analyte.

Exemplary light-transducing protein that alters binding to a target analyte include, but are not limited to improved light-induced dimer (iLID) which binds to SspB—see for example Guntas et al. https://doi(dot)org/10(dot)1073/pnas(dot)1417910112, the contents of which is incorporated herein by reference. Other exemplary light-transducing proteins that alter binding to a target analyte include LOVETRAP™ (see for example optobase(dot)org/switches/LOV-domains/AsLOV2/LOVTRAP) and TULIP™ (see for example optobase(dot)org/switches/LOV-domains/AsLOV2/TULIP/).

The isolating method of this aspect of the present invention may be carried out in biological fluids, including, but not limited to whole blood, serum, urine, cerebrospinal fluid, semen and saliva.

Removal of non-bound components may be carried out by passing the solution over a column (e.g. resin), wherein the resin is attached to the fusion protein. The analyte will be captured by the resin and non-relevant components present in the solution will flow-through.

In another embodiment, removal of the non-bound components may be carried out by contacting the solution with beads (e.g. magnetic bead), wherein the beads are coated with the fusion protein. The analyte will be captured by the beads. The beads may be separated from the solution (e.g. using a magnetic force, or by centrifugation) and non-relevant components present in the solution can then be removed.

Another use of the fusion protein described herein is for targeting to particles which have a cognate moiety on the surface thereof which is capable of binding to the light transducing moiety of the fusion protein. Thus, the fusion protein described herein may be targeted to the outer surface of particles in a light inducible manner.

Thus, according to another aspect of the present invention there is provided a method of targeting the composition of matter described herein to a particle having an outer surface, wherein a binding moiety (e.g. SspB) is attached to said outer surface of the particle, the binding moiety being capable of binding to the light transducing protein (e.g., iLID) upon emission of photons by the light generating protein, the method comprising contacting the composition of matter with a substrate that is capable of inducing emission of a photon by said light-generating protein under conditions which promote binding of said composition of matter to the particle via the binding moiety.

Exemplary particles of this aspect of the present invention include biological cells and synthetic cells (as further described herein above).

The synthetic particles may comprises a therapeutic or diagnostic agent

Targeting may be carried out in vivo or ex vivo.

As illustrated in FIGS. 1F and 1G, the phospholipid membrane is capability of protect DNA from radiation induced-damage. Moreover, the lipid composition was shown to modify the light transmission through the membrane. Therefore, light intensities going in and out of synthetic cells can be tuned by selecting the appropriate membrane components. Thus, the present inventors conceive of topical compositions comprising a membrane of lipids which reduces penetration of ultraviolet light and allows penetration of visible light.

Examples of lipids have been described herein above.

In one embodiment, the membrane of lipids comprise phospholipids.

In one embodiment, the lipids are formed in a lipid bilayer, in another the lipids are formed in a lipid monolayer.

In one embodiment, the lipids have a chain length of at least 16 carbons and wherein at least one of the 16 carbons is unsaturated e.g. -α-phosphatidylcholine, hydrogenated (Soy), (HSPC), 1,2-dioleoyl-sn-glycero-3-phosphocholine (DOPC) and 16:0,18:1 lipid 1-palmitoyl-2-oleoyl-glycero-3-phosphocholine (POPC).

In still another embodiment, the lipids of said lipid bilayer have a chain length of at least 12 carbons (e.g. 1,2-dimyristoyl-sn-glycero-3-phosphocholine (DMPC)).

In an embodiment of the presently claimed invention, the topical composition is present in the form of a liquid (e.g. eye-drops) cream, a foam, a lotion, a gel, a paste, a spray, a patch, a spray patch, a mousse or an ointment.

In an embodiment of the presently claimed invention, the topical composition further comprises at least one auxiliary agent selected form the group consisting of anti-wrinkle active agents, anti-acne active agents, emulsifiers, antioxidants, emollients, self-tanning active agents, skin lightening agents, sunscreen agents, UV absorbing agents, thickening agents, humectants, abrasives, absorbents, fragrances, buffering agents, opacifying agents, colorants, preservatives, fillers, pH adjusting agents and solvents.

In an embodiment of the presently claimed invention, the active agents are selected from the group consisting of anti-wrinkle agents like retinol, hyaluronic acid, ceramides, niacinamide, vitamin E, alpha hydroxy acids, anti-acne agents like clindamycin, benzamycin, benzoyl peroxide, and isotretinoin.

In an embodiment of the presently claimed invention, the UV absorbing agents or sun screen agents are selected from the group consisting of suitable sunscreening agents including, for example: p-aminobenzoic acid, its salts and its derivatives (ethyl, isobutyl, glyceryl esters; p-dimethylaminobenzoic acid, diethylaminohydroxybenzoylhexyl benzoate); anthranilates (i.e., o-aminobenzoates; methyl, menthyl, phenyl, benzyl, phenylethyl, linalyl, terpinyl, and cyclohexenyl esters); salicylates (amyl, phenyl, benzyl, menthyl, glyceryl, and dipropyleneglycol esters); cinnamic acid derivatives (menthyl and benzyl esters, butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); dihydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); hydroxy- or methoxy-substituted benzophenones; uric and vilouric acids; tannic acid and its derivatives (e.g., hexaethylether); (butyl carbotol) (6-propyl piperonyl) ether; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; and 4-isopropyl-di-benzoylmethane. In a preferred embodiment of the presently claimed invention, the UV absorbing agents or sun screen agents are selected from the group consisting of 2-ethylhexyl-p-methoxycinnamate, 4,4′-t-butyl methoxydibenzoyl-methane, 2-hydroxy-4-methoxybenzophenone, octyldimethyl-p-aminobenzoic acid, digalloyltrioleate, 2,2-dihydroxy-4-methoxybenzophenone, ethyl-4(bis(hydroxypropyl)) aminobenzoate, 2-ethylhexyl-2-cyano-3,3 -diphenylacrylate, 2-ethylhexylsalicylate, glyceryl-p-aminobenzoate, 3,3,5-trimethylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, Methylene Bis-benzotriazolyltetramethylbutylphenol and mixtures thereof.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization-A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Lipids: 1,2-dioleoyl- sn-glycero-3 -phosphocholine (DOPC), 1-palmitoyl-2-oleoyl-sn-glycero-3-phosphocholine (POPC), 1,2-dimyristoyl-sn-glycero-3-phosphocholine (DMPC), 1,2-dipalmitoyl- sn-glycero-3 -phosphocholine (DPPC), L-α-phosphatidylcholine, hydrogenated (Soy) (HSPC), 1,2-dioleoyl-sn-glycero-3-phosphoethanolamine (DOPE) and 1,2-dioleoyl- sn-glycero-3 -phospho-(1′-rac-glycerol) (sodium salt) (DOPG) were purchased from Lipoid (Germany). 1,2-dioleoyl-sn-glycero-3-[(N-(5-amino-l-carboxypentyl) iminodiacetic acid) succinyl] (nickel salt) (DGS-NTA(Ni)) was purchased from Avanti Polar Lipids (USA). Cholesterol was purchased from Sigma-Aldrich (Israel). DOPE-PEG4-biotin and DOPE-cy5 were synthesized by reacting DOPE with NHS-PEG4-biotin or NHS-cy5 (BDL pharma,

China).

Liposome preparation and absorbance measurements: Liposomes were prepared using the ethanol injection method. Lipids were weighed and dissolved in absolute ethanol and subsequently injected into calcium-free Dulbecco' s phosphate buffer saline (PBS; Sigma-Aldrich) preheated to 65° C. reaching a final lipid concentration of 50 mM. The liposomes were extruded five times using a high-pressure Lipex extruder (Northern Lipids, Canada) through 400, 200, and 100 nm polycarbonate-etched membrane (Whatman, Newton, Mass., USA) at 65° C. For the absorbance measurements, liposomes were diluted 50-fold and measured in a UV-star 96-well microplate (Greiner bio-one, Germany) using the Infinite 200PRO multimode reader (TECAN, Switzerland).

Synthetic Cell Preparation

Preparation of bacterial lysate for synthetic cell solutions: S30 bacterial lysate was prepared as described previously from BL21(DE3) E. coli transformed with the T7 polymerase expressing TargeTron vector pAR1219⁴⁷. For Gaussia luciferase expressing synthetic cells, DTT and β-mercaptoethanol were excluded from the S30 solution during the lysate preparation.

Emulsion transfer method: Synthetic cell preparation using the emulsion transfer method was performed as previously described with several modifications⁴⁷.

Shaking method: Synthetic cell preparation using the shaking method was performed as previously described by Gopfrich, et al³⁹. 6mM of 100 nm liposomes were prepared using the thin film hydration method. A thin lipid film of POPC, DOPG, DGS-NTA(Ni), DOPE-PEG4-biotin and DOPE-Cy5 at molar ratios of 73.5:20:5:1:0.5 was hydrated with PBS containing 10 mM MgCl₂ and extruded as described in the liposomes' preparation section. 100 μl of liposomes diluted to 2 mM were mixed with 200 μl of FC-40 oil (FL-0005-HP; Iolitec, Germany) with 10 mM of PFPE-carboxylic acid (Krytox; Costenoble, Germany) and 0.8% of fluorosurfactant (RAN Biotechnologies, USA) and incubated overnight. The bottom layer was subsequently removed and 100 μl of PBS, followed by 100 μl of 1H,1H,2H,2H-Perfluoro-1-octanol (PFO; Sigma-Aldrich) were added on top of the remaining top layer. After 40 minutes of incubation, the top layer was extracted, containing the synthetic cells.

UV exposure induced DNA damage: 100 μg ml⁻¹ of DNA in PBS or DNA encapsulated in synthetic cells were exposed for 20 minutes to 254 nm UV light (R-52 Grid lamp; UVP, USA) placed 10 cm above a 96-well plate. The exposed free DNA was subsequently encapsulated in synthetic cells. DNA from the samples exposed to UV before and after encapsulation was extracted and PCR amplified using Phusion polymerase (Thermo-fisher, USA). The reaction products were then blotted on a 1% agarose gel.

Luminescence Assays

Luciferase Production kinetics: Gluc expressing synthetic cells were incubated at 37° C. At each time point, 15 μl of synthetic cells were taken and diluted 4 fold in PBS. 25 μl of the diluted sample and 25 μl of native coelenterazine (Nanolight, USA) in a final concentration of 2.5 μM were added to a 384-well white microplate. Luminescence was measured using the Infinite 200PRO multimode reader (TECAN).

Luciferase kinetics and re-activation of light emission: Gluc-expressing synthetic cells were incubated at 37° C. for 1 hour. For measuring the luciferase reaction kinetics, the synthetic cells were subsequently diluted by 400 folds and 75 μl of synthetic cells were mixed with 75 μl of native CTZ at a final concentration of 100 μM in a 96-well white microplate. Luminescence was measured every 30 seconds for 15 minutes. For luminescence re-activation measurements, the synthetic cells were diluted 4 folds and 25 μl of synthetic cells were mixed with 25 μl of native CTZ at a final concentration of 2.5 μM in a 384-well white microplate. After measuring luminescence in one-minute intervals for 5 minutes, 25 μl of 5 μM native CTZ were added to the same well and luminescence was measured again for 5 minutes.

IVIS luminescence measurements: Light emission measurements using the IVIS SpectrumCT Pre-Clinical In-Vivo Imaging System (PerkinElmer, USA) were performed in luminescence mode with exposure time 0.2 sec, binning factor 2 and f Number 8. All images were analyzed using the LivingImage software.

Synthetic cell size and concentration measurements: Size analysis of synthetic cells was performed by light diffraction using the Mastersizer 3000 (Malvern Instrument, UK). The synthetic cell concentration was measured by flow cytometry using the AMNIS ImageStream®^{X} Mk II (Luminex Corporation, USA). Synthetic cells were diluted 10 folds in the synthetic cells' outer solution and counted by dividing the number of events verified as synthetic cells using the images in the brightfield channel and the total volume analyzed by the device.

CryoSEM imaging: Cryo-SEM imaging of synthetic cells was performed using a ZeissUltra Plus HR-SEM equipped with a Bal -Tec cryo-stage. The cells were mounted on a cryoSEM sample holder, plunged into a freezing liquid ethane bath and then transferred to a liquid nitrogen bath. Then, both sample holder and specimen were transferred to a VT-100 shuttle (Leica, USA) under low temperature and vacuum conditions. The shuttle was connected to a Leica EM BAF 060 Freeze-Fracture replication and cryo-SEM sample preparation system to expose a fracture profile of the specimen with a blade. The sample was then transferred in the VT-100 shuttle to the pre-cooled HR-SEM chamber for imaging.

Protein expression and purification: BL21(DE3) E. coli (NEB) were used for the expression of DsbC-his, EL222-his, iLID and his-maltose binding protein (MBP)-mRFP1-sspB-Nano. Expression of Gluc-EL222-his and his-Gluc-iLID was performed in SHuffle T7 E. coli. A 5 ml Luria-broth starter culture for each protein was incubated overnight at 37° C. and 250 rpm with the compatible antibiotics (ampicillin at 100 μg m1⁻¹ or kanamycin at 25 μg ml⁻¹). The starter was then transferred to 500 ml of Terrific-broth supplemented with antibiotics in the same concentration and grown at 37° C. and 250 rpm to optical density (OD) of 0.5, when they were induced with 500 μM of Isopropyl β-D-1-thiogalactopyranoside (IPTG). DsbC-his and EL222-his were incubated at 37° C. and 250 rpm following induction until reaching an OD of ˜4. His-iLID, his-MBP-mRFP1-sspB-Nano, Gluc-EL222-his and his-Gluc-iLID were grown at 16° C. and 250 rpm until reaching similar OD values. Cells were harvested by centrifugation at 7,000×g for 10 minutes at 4° C. and kept at −20° C. until to the next step.

For protein purification, the pellet was resuspended in PBS (in the case of DsbC-his and EL222-his), 50 mM Tris, 300 mM NaCl, pH 7.4 (in the case of his-iLID and his-MBP-RFP-sspB-Nano) or 300 mM NaCl, 50 mM phosphate buffer, pH 8.0 (in the case of his-Gluc-iLID and Gluc-EL222-his). The cells were fractionated by two passes through an emulsiFlex-C3 high pressure homogenizer (Avestin, Germany) and the lysate was spun down two times for 15 minutes at 20,000×g. The supernatant was passed through an AKTA purifier chromatography system (Cytiva, USA) using a HisTrap HP 5 ml column and eluted with elution buffer with similar composition to the loading buffer supplemented with 500 mM imidazole. The protein containing fractions were dialyzed in a 12-14 kD membrane (Spectrum Laboratories, USA) against their original resuspension buffer.

To remove the his-MBP domain from the mRFP1-SspB-Nano protein, the eluted MBP-RFP-sspB proteins were cut with TEV protease (NEB) using a digestion site between the MBP and the RPF sections. 300 μg of his-MBP-RFP-sspB-Nano were diluted to a total reaction volume of 880 μl. 20 μl of TEV Protease Reaction Buffer (10×) and 100 μl of TEV Protease were added, then incubated at 4° C. overnight. 10 reactions were samples were pooled together and passed through a Ni Sepharose 6 Fast Flow histidine-tagged protein purification resin (Cytiva). The flow-through containing the RFP-SspB-Nano protein, was collected and concentrated using Amicon ultra 15 kDa (Merck, USA). The proteins were dialyzed overnight in PBS.

Western blot Analysis: Cell free protein synthesis reactions and synthetic cell samples after protein production were analyzed using SDS-PAGE with a 12% acrylamide gel. The gel was blotted onto a nitrocellulose membrane and blocked with 5% nonfat milk powder in Tris□buffered saline. Gaussia luciferase Polyclonal Antibody (Invitrogen, USA) diluted 1:3750 in Tris□buffered saline with 0.5% Tween□ 20 and 0.5% nonfat milk powder was incubated with the membrane overnight at 4° C. After washing, the blots were incubated with horseradish peroxidase□conjugated anti□ rabbit (goat origin) secondary antibody (GenScript, USA) diluted to 1:20,000 and developed with Clarity Western ECL Blotting Substrate (BioRad, USA). The results were imaged using the Fusion FX6 imaging system (Vilber, France).

For quantification of Gluc production, analysis of the images was performed with ImageJ gel analysis plug-in and use of a calibration curve for Gluc in known concentrations.

Activation of Photoconidiation in Fungi by Synthetic Cells

Fungal growth: A Trichoderma atroviride inoculum, was plated on the center of a PDYC (24 g l⁻¹ potato dextrose broth (Difco, UK), 1.2 g l⁻¹ casein hydrolysate (Sigma-Aldrich), 2 g l⁻⁻¹ yeast extract (Difco) agar plate and incubated for twenty-four hours in dark conditions. 3 ml of PDYC media were subsequently added to a new 10 cm culture plate. In the center of the plate a Whatman 50 filter paper cut to a diameter of 9 cm was placed over an 8 cm Whatman 1 filter paper. On top of these, a 0.5 cm square from the periphery of the fungal growth on the agar plate was placed in the center and incubated in the dark at room temperature for 48 hours.

Exposure to synthetic cells: Synthetic cells were produced using the emulsion transfer method and diluted to the desired cell concentration. Two consecutive exposures of the fungi to synthetic cells were performed. In each exposure, 500 μl of synthetic cells were mixed with 500 μl of native CTZ in a final concentration of 100 μM in a transparent plastic bag. The bag was localized over the periphery of the fungal colony on the top part of the plate for 15 minutes and then removed. The plates were left for incubation in the dark for an additional period 24 hours, after which they were imaged using a regular camera.

Image analysis: Background normalization of the images was performed manually in ImageJ to achieve the same average pixel value for all images⁴⁸. The images were then converted to grayscale and then to black and white using a pixel threshold value equal to 45. The percentage of black pixels in a rectangle containing the top part of the plate where the synthetic cells were placed was calculated separately for each image.

Membrane Recruitment in Synthetic Cells

Membrane recruitment of RFP-sspB-Nano: A PDMS-walled chamber (Sylgard 184; Dow, USA), 5.5 mm in diameter and 2.5 mm height, was placed on a 22×50 mm deckglaser glass slides (Marienfeld, Germany). The bottom of the chamber was coated with 10 μg ml⁻¹ streptavidin (Promega) overnight at 4° C., or with 1% bovine serum albumin (Sigma-Aldrich) for 1 hour at room temperature. The chamber was washed with PBS and in case of streptavidin coating, was coated once more for 1 hour with 5 mg ml⁻¹ of cold water fish skin gelatin (Sigma-Aldrich) and washed again with PBS.

Synthetic cells were prepared using the shaking method (see synthetic-cell preparation), and 100 nM of his-iLID or his-Gluc-iLID were added to the solution and incubated for 1 hour at room temperature and shaking of 100 rpm. Under dark conditions, 25 nM of RFP-sspB-Nano were added to the cells and placed in the coated chamber in dark conditions for 30 minutes.

Imaging was performed using a standard inverted microscope (Eclipse Ti2; Nikon) outfitted with a 60× 1.4 NA objective lens. Low intensity laser illumination of 0.01 mW 640 nm and 1 mW 561 nm lasers for imaging of Cy5 and mRFP1 respectively were used. Images were captured on a Sona sCMOS detector (Andor, Northern Ireland). Blue light laser illumination was performed with 488 nm laser at 0.1 mW with on-off intervals of 1.25 and 3.75 seconds for one minute. For recruitment in synthetic cells functionalized with his-Gluc-iLID, native coelenterazine (0.2 nmol) was added every minute for a total of four minutes.

Gluc-iLID light emission imaging: Synthetic cells functionalized with Gluc-iLID were imaged after addition of 100 μM substrate (final concentration) using a standard inverted microscope (Eclipse Ti2; Nikon) outfitted with a 40× 0.75 NA objective lens (Nikon) and equipped with iXON EMCCD camera (Andor). Images were obtained with 400 msec exposure time and gain 300.

Transcription Activation in Synthetic Cells

LED illumination system: Five 470 nm blue LEDs (C503B-BAN-CY0C0461; Mouser, Israel) were connected together in series using an Arduino microcontroller and an external power supplier 0-30 V. On-off intervals were set by controlling relay modules using the Arduino IDE software. Blue light intensity was measured with a S310C light sensor (Thorlabs, USA).

EL222 mediated activation of transcription in cell free reactions: Cell-free reactions based on the internal synthetic cell solution for Rluc expression or E. coli S30 extract system for circular DNA (Promega) for RFP expression were supplemented with 2.5 μM EL222 (unless stated otherwise) and incubated at 37° C. in 384-well microplates coated with an adhesive film to prevent evaporation. LEDs were placed 7 cm above the plate, providing approximately 12 W M⁻², with on-off intervals of 20 and 70 seconds. For analysis of Rluc expression, native CTZ was added to a final concentration of 5 μM and luminescence was measured. For analysis of RFP expression, fluorescence intensity was measured with excitation/emission at 540 nm/600 nm.

EL222 activation in Synthetic cells: Synthetic cells with an internal solution based on the E. coli S30 extract system for circular DNA (Promega) were supplemented with 2.5 μM EL222. Prior to their incubation, each synthetic cell batch was divided into dark and light groups, both incubated at 37° C. in 384-well microplates coated with an adhesive film under dark or blue-light conditions. LEDs were placed 2.8 cm above the plate, providing approximately 19 W m⁻², with on-off intervals of 20 and 70 seconds. Self-activating synthetic cells based on the E. coli S30 extract system for circular DNA (Promega) were supplemented with 2.5 μM of Gluc-EL222. Prior to their incubation, each synthetic cell batch was divided into dark and light groups, both incubated at 37° C. Native CTZ (0.2 nmol) was added every 30 minutes to the light group for a total of four times. The samples were further incubated for 90 more minutes after the last substrate addition. Just prior to the final fluorescence measurements, CTZ was added to the dark group in the same concentration that was added to the light group in order to eliminate differences due to substrate auto-fluorescence.

Results

Optimizing the Lipid Composition of Light-Interacting Synthetic Cells

The phospholipid membrane is the main physical barrier for light emission or light absorption in synthetic cells composed of giant unilamellar vesicles (GUVs), and must therefore have favorable optical properties. Hence, the first step in the design of light-generating and light-responding synthetic cells is to carefully design their membrane composition.

Factors affecting blue light transmission through the lipid membrane were investigated and the phospholipid composition of the membrane was altered accordingly to allow maximal light transfer with minimal losses. First, the absorbance of 480 nm light by 100-nm liposomes composed of a single phospholipid type was measured (FIG. 1B). 100-nm liposomes were selected for this measurement due to their lower polydispersity index, thereby reducing sample-to-sample inaccuracies originating from size-dependent light interactions (i.e. light scattering). The lipid-light interactions demonstrated a correlation between the phospholipid tail length and light absorbance, which increased in accordance to increase in the size of the phospholipid tail (FIG. 1B). L-α-phosphatidylcholine, hydrogenated (Soy), (HSPC) which is composed of 88.6% of 18:0 fatty acids had the highest absorbance, followed by 1,2-dipalmitoyl-sn-glycero-3-phosphocholine (DPPC) with 16:0 tails and 1,2-dimyristoyl-sn-glycero-3-phosphocholine (DMPC) with 14:0 tails. This trend was also apparent in liposomes composed of unsaturated phospholipids, in which the 18:1 lipid 1,2-dioleoyl-sn-glycero-3-phosphocholine (DOPC) demonstrated higher light attenuation compared to 16:0,18:1 lipid 1-palmitoyl-2-oleoyl-glycero-3-phosphocholine (POPC). Moreover, unsaturated lipids had lower absorbance in comparison to saturated-lipids-based liposomes with similar tail length.

While DMPC and POPC liposomes had the lowest measured light absorbance, the latter was selected as the main lipid in the synthetic cell due to its lower melting temperature (Tₘ) of −2 ° C. which enabled it to remain in a liquid phase during the synthetic cell production process performed at 4° C. Cholesterol was also added to the synthetic cell formulation (50% w/w ratio), despite its small contribution to light absorbance, in order to enhance membrane stability which is important, as the incubation for protein production is performed at 37° C.

Once the lipid composition of the membrane was selected, synthetic cells were produced using the emulsion transfer method. This method's simplicity, high encapsulation efficiency and high yield make it preferable for achieving high overall protein production²⁴. Light scattering measurements of the size distribution of the obtained cell population indicated a size distribution ranging from 1-200 μm with average D10, D50 and D90 values of 5.2, 18.6 and 51.8 μm, respectively (FIG. 1C). The cells' morphology was further validated with cryogenic scanning electron microscopy (cryo-SEM)—FIG. 1D.

The absorbance spectrum of different phospholipids was scanned at different wavelengths (230-800 nm) to understand if the trends recognized for 480 nm light were relevant for other wavelengths that could also be used for light signaling. The scan revealed a sharp increase in the absorbance of all the liposomal formulations in the UVC (200-290 nm) and UVB (290-320 nm) range (FIG. 1E). It was hypothesized that this result might hint that lipids played an evolutionary role as UV protecting agents in the prebiotic world which lacked an ozone layer and had high UV radiation levels. Thus, the ability of the phospholipid membrane to protect DNA from UV radiation damage using the present synthetic cell platform was examined (FIGS. 1F-G). Exposure of DNA to UV radiation is known to lead to the formation of pyrimidine dimers and other DNA photoproducts that damage the DNA functionality and lead to subsequent biological effects (FIG. 1F)^{25,26}. A 600-bp linear DNA oligonucleotide was exposed to UV radiation for 20 minutes before or after its encapsulation in a synthetic cell and the formation of DNA lesions was detected with polymerase chain-reactions (PCR). Encapsulation of the DNA in synthetic cell yielded evident PCR amplification indicating little UV-induced damage in comparison to the DNA that was irradiated prior to its encapsulation and yielded almost no product at all. This demonstrates an important attribute of phospholipids as UV-protecting agents.

Engineering Light-Generating Synthetic Cells

Synthetic cells capable of generating light were engineered by expressing luciferase enzymes—Renilla luciferase (Rluc) and a mutated variant of Gaussia luciferase (M43L, M110L)²⁸ sourced from the organisms Renilla reniformis and Gaussia princeps respectively. Both types of luciferase catalyze an ATP-independent bioluminescent oxidation reaction of the substrate coelenterazine (CTZ), conserving energy for the synthetic cell^{23,29}. In terms of light emission, both enzymes generate photons in the blue-range, suitable for Light-oxygen-voltage-sensing domain (LOV) domain activation. Nevertheless, while Rluc has rapid flash kinetics, the Gluc variant exhibits brighter and longer half-life of illumination (glow-like kinetics)²⁸.

Initial comparison of light production of both luciferase types in CFPS reactions with E. coli BL21(DE3) lysates indicated 2500-fold higher light emission in Rluc-expressing synthetic cells in comparison to weak illumination produced from the Gluc-expressing synthetic cells (FIG. 2A). The poor production of Gluc in the synthetic cells was due to misfolding of its five disulfide bonds in the reducing environment of the synthetic cells that contains both 1,4-Dithiothreitol (DTT) and 2-mercaptoethanol³⁰. Moreover, oxidative folding for disulfide bond generation in E. coli is performed in the periplasm and not in the reducing environment of the bacterial cytoplasm which is a main component of the lysate³¹. Therefore, the synthetic cells' internal composition was altered. Reducing agents DTT and 2-mercaptoethanol were excluded from the internal solution, and glutathione and disulfide bond isomerase C (DsbC) from E. coli were added to improve Gluc folding³². A range of oxidized and reduced glutathione concentrations were added to the inner solution of the synthetic cells and light emission after Coelenterazine (CTZ) addition was measured. 4 mM of oxidized and 1 mM of reduced glutathione were found to be optimal for Gluc production with the BL21(DE3) lysate. After further addition of 75 μg ml⁻¹ of DsbC to the glutathione-supplemented synthetic cell internal solution, light emission from Gluc-expressing synthetic cells was 5-fold higher than that of the Rluc-expressing synthetic cells (FIG. 2B), and was easily visible by eye in solution (FIG. 2C). Gluc expression in synthetic cells was quantified with Western blot analysis demonstrating production of 43 ng μl⁻¹ (FIG. 2D) of protein. This translates to an average of 25 pg of Gluc per cell, considering a synthetic cell concentration of 1.682·10⁶ cells ml1⁻¹, as quantified using multispectral imaging flow cytometry (see methods).

The protein production and light emission properties of Gluc-expressing synthetic cells were further characterized under different conditions. Protein production kinetics in synthetic cells at 37° C. were monitored using light-emission assays performed at different incubation times (FIG. 2E). Gluc levels increased over the first 60 minutes and plateaued at 90 minutes. Light emission kinetic measurements demonstrated a t_{1/2} of two minutes for synthetic cells incubated with 100 μM of CTZ (FIG. 2F). After 15 minutes, the illumination intensity was approximately 7% of the initial intensity measured and still more than 100,000 fold higher than the intensity measured from control synthetic cells without a DNA template encoding for luciferase. Next, the dependency of light intensity on the synthetic cell concentration was demonstrated (FIG. 2G). A concentration of 420,000 synthetic cells ml⁻¹ was found to generate the maximal light intensity upon CTZ addition and was used for the subsequent experiments. In comparison, 2 and 4-times higher cell concentrations and 2.5 and 5-times lower cell concentrations had significantly lower light emission. Increasing concentrations of synthetic cells increase the total Gluc concentration, but also the total phospholipid and cholesterol concentration which have opposite contributions to the light intensity. The yield of emitted light, therefore, is balanced between these two parameters. Light intensity was also controlled by altering the substrate concentration, and changed by an order of magnitude from 10¹¹ photons s⁻¹ cm⁻² sr⁻¹ with 10 μM CTZ to 10¹² photons s⁻¹ cm⁻² sr⁻¹ with 200 μM CTZ (FIG. 2H). Lower light intensities were also detectable using lower concentrations of substrate, down to 10 nM of CTZ. Temporal control of illumination was achieved by timed addition of the substrate to the solution. Synthetic cells produced more than a single light pulse, when an additional dose of substrate was supplied. The intensity of the light pulse generated upon addition of a second dose of substrate following the decay of the initial light emission reached intensities similar to those produced in the first illumination pulse (FIG. 21). This ability allows to maintain high light intensity levels when prolonged illumination is required.

Light-Producing Synthetic Cells Activate Fungal Cells

Intercellular signaling between light-producing synthetic cells and light-responsive natural cells was established using the soil fungus Trichoderma atroviride. Two blue-light regulator (BLR) proteins control the photo-activation of this fungus in response to blue light, triggering several downstream processes including conidiation and the expression of the DNA repairing photolyase enzyme³³⁻³⁵. Photo-activation was initially tested with externally-supplied blue light to ensure the fungus' ability to induce conidiation. T. atroviride were plated and grown in dark room conditions for 48 hours. 24 hours after exposure to LED-sourced blue light (15 mW cm⁻²) for 1 minute, a peripheral ring of spores was evident on the border of the fungi colony. Synthetic cell illumination was supplied in two consecutive induction rounds, each with 500 μL of synthetic cells supplemented with 100 μM CTZ that were added to a restricted section in the fungi plate and incubated for 15 minutes (FIG. 3A). After a second incubation period of 24 hours in the dark following induction, conidiation was quantified by calculating the percentage of sporulated area out of the total area exposed to synthetic cells (FIGS. 3B-C). Fungi incubated with Gluc-expressing synthetic cells demonstrated an average of 7.3% sporulated area, a 19-fold increase in comparison to fungi incubated with synthetic cells without a DNA template (FIG. 3C).

The effect of the synthetic cells' concentration on the activation of the BLR pathway was subsequently demonstrated. A range of synthetic cell dilutions were incubated on the T. atroviride plates, as performed in the initial experiment, and sporulation was quantified (FIG. 3D). A minimal concentration of 84,000 synthetic cells ml⁻¹ was required for activation, with a ˜1.7-fold increase in sporulated area observed between 84,000 cells ml⁻¹ and 420,000 cells ml⁻¹. In a previous study, Horwitz et al.³³ showed that approximately 10¹⁹ photons m⁻² of broadband blue light were necessary for initial activation of the T. atroviride culture in similar experimental conditions. This provides an approximate measure of the total photon flux produced by the synthetic cells during the experiment. The observed synthetic cell concentration-dependent response of T. atroviride resembles chemical quorum sensing mechanisms in other species, yet here the sensing is based on light dosage and can occur even when the synthetic and natural cells are separated by a physical barrier.

Bioluminescence-Activated Transcription in Synthetic Cells

Next, auto-activation of two different cellular processes in synthetic cells was demonstrated using bioluminescent reactions: induction of protein expression and membrane recruitment.

Control over protein expression was performed using the light-inducible bacterial transcription factor EL222, that dimerizes when exposed to blue light and binds to a specific region in the pBLind promoter to initiate transcription (FIG. 4A)^{36,37}. To simplify the integration of EL222 into the synthetic cell inner solution, calibration of the required EL222 concentration was initially performed in CFPS reactions (before encapsulation in synthetic cells) using an external LED blue light source for activation. Rluc was used as a reporter protein, and a DNA plasmid containing its reading frame after the pBLind promoter was prepared. Purified EL222 was added in concentrations of 2.5 μM, 5 μM and 10 μM to the CFPS mix and incubated for 1 hour in dark or light conditions (FIG. 4B). Rluc expression levels were analyzed by adding CTZ and measuring luminescence. EL222 had the most significant light-to-dark ratio at 2.5 μM, and demonstrated significant light-to-dark differences at 5 and 10 μM. Production of monomeric RFP (mRFP1), that is characterized by a longer folding time, in CFPS reactions with 2.5 μM EL222 was tested subsequently (FIG. 4C). Elevated mRFP1 levels were evident in the reaction mix containing EL222 and mRPF1 DNA after 5 hours of incubation with blue-light, in comparison to the same solution incubated in dark conditions, and continued to increase through the 12 hours measured (FIG. 4C, filled circles). Some increase in signal was evident in the dark-incubated sample without DNA (FIG. 4C, hollow black circles), and is associated with increase in auto fluorescence over time.

After establishing the reaction conditions in CFPS, the EL222 system was integrated in synthetic cells and expression of Rluc using an external LED light was activated. The higher light absorbance of synthetic cells compared to CFPS required an increase in the light intensity used for activation, while avoiding overheating that might lead to protein denaturation. Therefore, the light intensity was increased from approximately 12 W m⁻² to 19 W m⁻² with on-off intervals of 20 and 70 seconds respectively. Under these conditions, synthetic cells encapsulating EL222 monomers and a DNA plasmid expressing Rluc under the pBLind promoter showed 2.4-fold increase in Rluc expression in light vs. dark conditions (FIG. 4D). In comparison, synthetic cells with EL222 and no DNA or a DNA plasmid with Rluc expressed under the viral T7 promoter which is not specific to EL222, showed negligible changes.

According to the light intensity requirements for synthetic cell activation used in the previous experiments, it was evident that the light generated from the Gluc-expressing synthetic cells would not be sufficient for EL222 activation. In order to activate EL222 with bioluminescence, close proximity between the light source and the responsive protein is required. To achieve this, a new fusion protein was designed, with Gluc on its N-terminal end connected through a flexible peptide linker to EL222 on its C-terminal end. This construct utilizes bioluminescence resonance energy transfer (BRET) to exploit the energy from the bioluminescent luciferase reaction on its substrate CTZ, to directly activate the target protein (FIG. 4E). The Gluc-EL222 protein was added to the synthetic cell interior instead of the native EL222, and followed mRFP1 production with and without the addition of CTZ (FIG. 4F). A 3.2-fold increase in mRFP1 production was observed when CTZ was added to the synthetic cells in comparison to synthetic cells that were incubated in the dark without CTZ. This demonstrates the functionality of the fusion protein as a self-activating transcription factor in synthetic cells.

Bioluminescence-Activated Membrane Recruitment in Synthetic Cells

To further explore the potential of self-activating synthetic cells, an additional mechanism of bioluminescence-induced membrane recruitment was engineered. This was performed using a hetero-dimerization reaction with one monomer localized to the synthetic cell membrane (iLID) and the other free in solution (sspB-Nano)³⁸. DGS-NTA-Ni lipids were incorporated into the synthetic cell membrane to bind his-tagged iLID and the localization of mRFP1 fused to the sspB-Nano protein was monitored. In these experiments, the synthetic cells were prepared with the shaking method, which is based on charge-mediated liposome fusion inside surfactant-stabilized droplets and enables higher variability in lipid selection in comparison to the emulsion-transfer method³⁹. This allowed to prepare synthetic cells with a membrane composition of 73.5% POPC, 20% DOPG, 5% DGS-NTA-Ni, 1% DOPE-PEG4-biotin and 0.5%

DOPE-Cy5.

As in the previous process of EL222 activation, Gluc-expressing CFPS reactions did not provide sufficient light to activate the LOV domain of the iLID and initiate sspB-Nano recruitment to the synthetic cell membrane. A second fusion protein was engineered, N-terminal Gluc fused to C-terminal iLID with a linker peptide and an N-terminal his-tag (FIG. 5A). This structure facilitates binding of the fusion protein to the synthetic cell membrane from the Gluc end and exposes the iLID to the extracellular environment with lower steric interference (FIG. 5A). Gluc-iLID binding to the synthetic cells membrane was verified by imaging of the synthetic cells after addition of CTZ (FIG. 5B). Bioluminescence emission localized to the synthetic cells' membrane was evident when imaging without laser excitation, and validated the binding and activity of Gluc.

The activation of membrane recruitment in individual synthetic cells was demonstrated using fluorescent microscopy. In order to enable single cell tracing under mixing conditions due to substrate additions, a biotinylated lipid (DOPE-PEG4-biotin) was added to the synthetic cell membrane and the microscope slides were coated with streptavidin. Synthetic cells remained bound to the surface even after multiple additions and mixing of substrate. Activation of iLID and Gluc-iLID was tested using either 488 nm laser or by addition of CTZ. The normalized mRFP1 intensity in synthetic cells with membrane-bound Gluc-iLID increased by 2.5-fold after 4 additions of CTZ in comparison to the initial dark conditions (FIG. 5C, triangles). Synthetic cells with membrane-bound iLID (without Gluc) were not activated by addition of CTZ (FIG. 5C, circles), but demonstrated a 1.5-fold increase in mRFP1 signal after 4 minutes of blue laser activation (FIG. 5C, squares).

The difference in the activation levels between iLID and Gluc-iLID synthetic cells can be associated with the addition of Gluc to the N-terminal side, that placed the iLID protein further away from the membrane and increased its exposure to the external solution. It was previously shown that binding of iLID to a membrane reduced its sspB-Nano binding ability in comparison to unbound iLID due to steric hindrance. Hence, the addition of Gluc which also works as an anchor improved this is sue³⁸. This was further validated by activation of Gluc-iLID with blue laser light, which resulted in an even higher increase in mRFP1 intensity, with more than 8-times difference between dark and light conditions. Taken together, these results demonstrate efficient and controlled bioluminescence-activated membrane recruitment of sspB -tagged proteins to Gluc-iLID labeled synthetic cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

1. Gopfrich, K., Platzman, I. & Spatz, J.P. Mastering Complexity: Towards Bottom-up Construction of Multifunctional Eukaryotic Synthetic Cells. Trends in biotechnology (2018).
2. Noireaux, V., & Libchaber, A. A vesicle bioreactor as a step toward an artificial cell assembly. Proceedings of the National Academy of Sciences 101, 17669-17674 (2004).
3. Fanalista, F. et al. Shape and size control of artificial cells for bottom-up biology. ACS nano 13, 5439-5450 (2019).
4. Elani, Y., Law, R. V. & Ces, O. Protein synthesis in artificial cells: using compartmentalisation for spatial organisation in vesicle bioreactors. Physical Chemistry Chemical Physics 17, 15534-15537 (2015).
5. Chen, Z. et al. Light □Gated Synthetic Protocells for Plasmon□Enhanced Chemiosmotic Gradient Generation and ATP Synthesis. Angewandte Chemie International Edition 58, 4896-4900 (2019).
6. Van Nies, P. et al. Self-replication of DNA by its encoded proteins in liposome-based synthetic cells. Nature communications 9, 1583 (2018).
7. Merkle, D., Kahya, N. & Schwille, P. Reconstitution and anchoring of cytoskeleton inside giant unilamellar vesicles. ChemBioChem 9, 2673-2681 (2008).
8. Chen, Z. et al. Synthetic beta cells for fusion-mediated dynamic insulin secretion. Nature chemical biology 14, 86 (2018).
9. Krinsky, N. et al. Synthetic cells synthesize therapeutic proteins inside tumors. Advanced healthcare materials 7, 1701163 (2018).
10. Blain, J.C. & Szostak, J.W. Progress toward synthetic cells. Annual review of biochemistry 83, 615-640 (2014).
11. Luisi, P. L., Ferri, F. & Stano, P. Approaches to semi-synthetic minimal cells: a review. Naturwissenschaften 93, 1-13 (2006).
12. Adamala, K. P., Martin-Alarcon, D. A., Guthrie-Honea, K. R. & Boyden, E. S. Engineering genetic circuit interactions within and between synthetic minimal cells. Nature chemistry 9, 431 (2017).
13. Lentini, R. et al. Integrating artificial with natural cells to translate chemical messages that direct E. coli behaviour. Nature communications 5, 4012 (2014).
14. Dupin, A. & Simmel, F. C. Signalling and differentiation in emulsion-based multi-compartmentalized in vitro gene circuits. Nature chemistry 11, 32-39 (2019). 15. Tang, T. D. et al. Gene-mediated chemical communication in synthetic protocell communities. ACS synthetic biology 7, 339-346 (2018).
16. Schroeder, A. et al. Remotely activated protein-producing nanoparticles. Nano letters 12, 2685-2689 (2012).
17. Bartelt, S. M., Steinkuehler, J., Dimova, R. & Wegner, S. V. Light guided motility of a minimal synthetic cell. Nano Letters (2018).
18. Berhanu, S., Ueda, T. & Kuruma, Y. Artificial photosynthetic cell producing energy for protein synthesis. Nature communications 10, 1-10 (2019).
19. Cardin, J. A. et al. Targeted optogenetic stimulation and recording of neurons in vivo using cell-type-specific expression of Channelrhodopsin-2. Nature protocols 5, 247 (2010).
20. Nussinovitch, U. & Gepstein, L. Optogenetics for in vivo cardiac pacing and resynchronization therapies. Nature biotechnology 33, 750 (2015).
21. Fan, F. & Wood, K.V. Bioluminescent assays for high-throughput screening. Assay and drug development technologies 5, 127-136 (2007).
22. Zoltowski, B. D., Vaccaro, B. & Crane, B. R. Mechanism-based tuning of a LOV domain photoreceptor. Nature chemical biology 5, 827-834 (2009).
23. Tannous, B. A., Kim, D.-E., Fernandez, J. L., Weissleder, R. & Breakefield, X. O. Codon-optimized Gaussia luciferase cDNA for mammalian gene expression in culture and in vivo. Molecular Therapy 11, 435-443 (2005).
24. Moga, A., Yandrapalli, N., Dimova, R. & Robinson, T. Optimization of the Inverted Emulsion Method for High□Yield Production of Biomimetic Giant Unilamellar Vesicles. ChemBioChem 20, 2674 (2019).
25. Kripke, M. L., Cox, P. A., Alas, L. G. & Yarosh, D. B. Pyrimidine dimers in DNA initiate systemic immunosuppression in UV-irradiated mice. Proceedings of the National Academy of Sciences 89, 7516-7520 (1992).
26. Hart, R., Setlow, R. & Woodhead, A. Evidence that pyrimidine dimers in DNA can give rise to tumors. Proceedings of the National Academy of Sciences 74, 5574-5578 (1977).
27. Ayala-Torres, S., Chen, Y., Svoboda, T., Rosenblatt, J. & Van Houten, B. Analysis of gene-specific DNA damage and repair using quantitative polymerase chain reaction. Methods 22, 135-147 (2000).
28. Welsh, J. P., Patel, K. G., Manthiram, K. & Swartz, J. R. Multiply mutated Gaussia luciferases provide prolonged and intense bioluminescence. Biochemical and biophysical research communications 389, 563-568 (2009).
29. Lorenz, W. W., McCann, R. O., Longiaru, M. & Cormier, M. J. Isolation and expression of a cDNA encoding Renilla reniformis luciferase. Proceedings of the National Academy of Sciences 88, 4438-4442 (1991).
30. Tannous, B. A. Gaussia luciferase reporter assay for monitoring biological processes in culture and in vivo. Nature protocols 4, 582 (2009).
31. Messens, J. & Collet, J.-F. Pathways of disulfide bond formation in Escherichia coli. The international journal of biochemistry & cell biology 38, 1050-1062 (2006).
32. Goerke, A. R., Loening, A. M., Gambhir, S. S. & Swartz, J. R. Cell-free metabolic engineering promotes high-level production of bioactive Gaussia princeps luciferase. Metabolic engineering 10, 187-200 (2008).
33. Horwitz, B. A., Perlman, A. & Gressel, J. Induction of Trichoderma sporulation by nanosecond laser pulses: evidence against cryptochrome cycling. Photochemistry and photobiology 51, 99-104 (1990).
34. Berrocal-Tito, G., Sametz-Baron, L., Eichenberg, K., Horwitz, B. A. & Herrera-Estrella, A. Rapid blue light regulation of a Trichoderma harzianum photolyase gene. Journal of Biological Chemistry 274, 14288-14294 (1999).
35. Casas-Flores, S., Rios-Momberg, M., Bibbins, M., Ponce-Noyola, P. & Herrera-Estrella, A. BLR-1 and BLR-2, key regulatory elements of photoconidiation and mycelial growth in Trichoderma atroviride. Microbiology 150, 3561-3569 (2004).
36. Motta-Mena, L. B. et al. An optogenetic gene expression system with rapid activation and deactivation kinetics. Nature chemical biology 10, 196-202 (2014).
37. Jayaraman, P. et al. Cell-free optogenetic gene expression system. ACS synthetic biology 7, 986-994 (2018).
38. Chervyachkova, E. et al. Dynamic blue light-switchable protein patterns on giant unilamellar vesicles. Chemical communications (2018).
39. Göpfrich, K. et al. One-Pot Assembly of Complex Giant Unilamellar Vesicle-Based Synthetic Cells. ACS synthetic biology (2019).
40. Lentini, R., Martin, N. Y. & Mansy, S. S. Communicating artificial cells. Current opinion in chemical biology 34, 53-61 (2016).
41. Aufinger, L. & Simmel, F. C. Establishing communication between artificial cells. Chemistry-A European Journal 25, 12659-12670 (2019).
42. Zhang, F. et al. Red-shifted optogenetic excitation: a tool for fast neural control derived from Volvox carteri. Nature neuroscience 11, 631-633 (2008).
43. Inoue, K. et al. Red-shifting mutation of light-driven sodium-pump rhodopsin. Nature communications 10, 1-11 (2019).
44. Chen, S. et al. Near-infrared deep brain stimulation via upconversion nanoparticle-mediated optogenetics. Science 359, 679-684 (2018).
45. Mattis, J. et al. Principles for applying optogenetic tools derived from direct comparative analysis of microbial opsins. Nature methods 9, 159 (2012).
46. Gong, X. et al. An ultra-sensitive step-function opsin for minimally invasive optogenetic stimulation in mice and macaques. Neuron 107, 38-51. e8 (2020).
47. Adir, O. et al. Preparing Protein Producing Synthetic Cells using Cell Free Bacterial Extracts, Liposomes and Emulsion Transfer. JoVE (Journal of Visualized Experiments), e60829 (2020).
48. Schindelin, J. et al. Fiji: an open-source platform for biological-image analysis. Nature methods 9, 676-682 (2012).

What is claimed is:

1. A composition of matter comprising a fusion protein attached to the outer surface of a substrate, wherein the fusion protein comprises a light-generating protein and a light-transducing protein.

2. A lipidated fusion protein comprising a light-generating protein and a light-transducing protein.

3. The composition of matter of claim 1, wherein the light-generating protein comprises an inducible light generating protein.

4. The composition of matter of claim 1, wherein said inducible light generating protein is a luciferase being optionally Gaussia luciferase or Renilla luciferase.

5. The composition of matter of claim 1, wherein said light transducing protein is improved light-induced dimer (iLID).

6. The composition of matter of claim 1, wherein the light-generating protein is attached to the light-transducing protein via a linker, preferably a linker that is a peptide.

7. The composition of matter of claim 1, wherein the fusion protein is lipidated.

8. The composition of claim 7, wherein the lipid of the lipidated fusion protein is attached to the light generating protein.

9. The composition of matter of claim 1, wherein the fusion protein further comprises a cell membrane targeting moiety.

10. The composition of matter of claim 9, wherein said cell comprises a synthetic cell.

11. The composition of matter of claim 1, wherein said substrate comprises a lipid particle.

12. The composition of matter of claim 1, wherein said lipid particle comprises a nickel chelating lipid and said light generating protein comprises a histidine tag.

13. A method of isolating an analyte comprising:
(a) contacting a solution which comprises the analyte with the composition of matter of claim 1, wherein the contacting is effected in the presence of a substrate that is capable of inducing emission of a photon by the light-generating protein; wherein the emission of the photon by the light generating protein is capable of inducing a response in the light-transducing protein that increases binding thereof to the analyte; and
(b) removimg non-bound components present in the solution, thereby isolating the analyte.

14. The method of claim 13, wherein said light-transducing protein is iLID.

15. A method of targeting the composition of matter of claim 1 to a particle having an outer surface, wherein a binding moiety is attached to said outer surface of said particle, said binding moiety being capable of binding to said light transducing protein upon emission of photons by the light generating protein, the method comprising contacting the composition of matter of claim 1 with a substrate that is capable of inducing emission of a photon by said light-generating protein under conditions which promote binding of said composition of matter to said particle, thereby targeting said composition of matter of claim 1 to said particle.

16. A topical composition comprising a membrane of lipids which reduces penetration of ultraviolet light and allows penetration of visible light.

17. The topical composition of claim 16, wherein said lipids have a chain length of at least 16 carbons and wherein at least one of said 16 carbons is unsaturated.

18. The topical composition of claim 16, wherein said membrane comprises a lipid bilayer or a lipid monolayer.

19. The topical composition of claim 18, wherein said lipids of said lipid bilayer have a chain length of at least 12 carbons.

20. The topical composition of claim 16, wherein said composition is formulated as eye drops or a sunscreen.


SEQUENCE LISTING (18 sequences)

SEQ ID NO: 1 - DNA, 510 bp, Artificial sequence
  Gaussia Luciferase (GLuc)
atgaaaccaacagaaaataacgaagattttaacatcgtcgcggtagcctcaaattttgcaaccaccgacttagacgcggatcgtgggaaattaccaggtaagaaattgccgttggaagtacttaaagaactggaagccaacgcacgcaaggctggatgcactcgtgggtgtttgatctgtctgtcgcatattaagtgtacgccaaagatgaaaaagttcatcccagggcgttgtcatacttacgaaggagacaaagagtcagcacagggcggaatcggggaagctatcgtagatatccctgagatcccaggcttcaaagacttggaaccgctggaacagtttattgctcaagttgacttgtgcgtcgactgtacgactgggtgccttaaagggctggcgaatgtccaatgtagtgatctgcttaagaagtggcttcctcagcgttgcgctacattcgcaagcaaaattcaggggcaagtggataagatcaaaggcgcgggtggtgactaa

SEQ ID NO: 2 - protein, 169 aa, Artificial sequence
  Gaussia Luciferase (GLuc)
MKPTENNEDFNIVAVASNFATTDLDADRGKLPGKKLPLEVLKELEANARKAGCTRGCLICLSHIKCTPKMKKFIPGRCHTYEGDKESAQGGIGEAIVDIPEIPGFKDLEPLEQFIAQVDLCVDCTTGCLKGLANVQCSDLLKKWLPQRCATFASKIQGQVDKIKGAGGD

SEQ ID NO: 3 - DNA, 459 bp, Artificial sequence
  iLID
atgggatccggggagtttctggcaaccacactggaacggatcgagaaaaatttcgtgattactgatccgagactgcctgacaacccaatcatttttgcgagcgattccttcctgcagctgacagaatattctcgggaagagatcctggggcgcaattgccgttttctgcagggacccgagacagaccgtgccactgttcggaaaatcagagatgctattgacaaccagactgaagtgaccgttcagctgatcaattataccaagagcggcaagaagttctggaacgtgttccacctgcagccgatgcgcgattataagggcgacgtccagtacttcattggcgtgcagctggatggcaccgaacgtcttcatggcgccgctgagcgtgaggcggtcatgctgatcaaaaagacagcctttcagattgctgaggcagcgaacgacgaaaattacttttaa

SEQ ID NO: 4 - protein, 152 aa, Artificial sequence
  iLID
MGSGEFLATTLERIEKNFVITDPRLPDNPIIFASDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVMLIKKTAFQIAEAANDENYF

SEQ ID NO: 5 - DNA, 678 bp, Artificial sequence
  EL222
atgttggatatgggacaagatcggccgatcgatggaagtggggcacccggggcagacgacacacgcgttgaggtgcaaccgccggcgcagtgggtcctcgacctgatcgaggccagcccgatcgcatcggtcgtgtccgatccgcgtctcgccgacaatccgctgatcgccatcaaccaggccttcaccgacctgaccggctattccgaagaagaatgcgtcggccgcaattgccgattcctggcaggttccggcaccgagccgtggctgaccgacaagatccgccaaggcgtgcgcgagcacaagccggtgctggtcgagatcctgaactacaagaaggacggcacgccgttccgcaatgccgtgctcgttgcaccgatctacgatgacgacgacgagcttctctatttcctcggcagccaggtcgaagtcgacgacgaccagcccaacatgggcatggcgcgccgcgaacgcgccgcggaaatgctcaagacgctgtcgccgcgccagctcgaggttacgacgctggtggcatcgggcttgcgcaacaaggaagtggcggcccggctcggcctgtcggagaaaaccgtcaagatgcaccgcgggctggtgatggaaaagctcaacctgaagaccagcgccgatctggtgcgcattgccgtcgaagccggaatctaa

SEQ ID NO: 6 - protein, 225 aa, Artificial sequence
  EL222
MLDMGQDRPIDGSGAPGADDTRVEVQPPAQWVLDLIEASPIASVVSDPRLADNPLIAINQAFTDLTGYSEEECVGRNCRFLAGSGTEPWLTDKIRQGVREHKPVLVEILNYKKDGTPFRNAVLVAPIYDDDDELLYFLGSQVEVDDDQPNMGMARRERAAEMLKTLSPRQLEVTTLVASGLRNKEVAARLGLSEKTVKMHRGLVMEKLNLKTSADLVRIAVEAGI

SEQ ID NO: 7 - DNA, 30 bp, Artificial sequence
  linker protein nucleic acid sequence
ggtggtggtggttcaggtggtggtggttca

SEQ ID NO: 8 - protein, 10 aa, Artificial sequence
  linker protein amino acid sequence
GGGGSGGGGS

SEQ ID NO: 9 - DNA, 1011 bp, Artificial sequence
  Gluc-iLID
atgcatcaccatcaccatcacaaaccaacagaaaataacgaagattttaacatcgtcgcggtagcctcaaattttgcaaccaccgacttagacgcggatcgtgggaaattaccaggtaagaaattgccgttggaagtacttaaagaactggaagccaacgcacgcaaggctggatgcactcgtgggtgtttgatctgtctgtcgcatattaagtgtacgccaaagatgaaaaagttcatcccagggcgttgtcatacttacgaaggagacaaagagtcagcacagggcggaatcggggaagctatcgtagatatccctgagatcccaggcttcaaagacttggaaccgctggaacagtttattgctcaagttgacttgtgcgtcgactgtacgactgggtgccttaaagggctggcgaatgtccaatgtagtgatctgcttaagaagtggcttcctcagcgttgcgctacattcgcaagcaaaattcaggggcaagtggataagatcaaaggcgcgggtggtgacggtggtggtggttcaggtggtggtggttcaggatccggggagtttctggcaaccacactggaacggatcgagaaaaatttcgtgattactgatccgagactgcctgacaacccaatcatttttgcgagcgattccttcctgcagctgacagaatattctcgggaagagatcctggggcgcaattgccgttttctgcagggacccgagacagaccgtgccactgttcggaaaatcagagatgctattgacaaccagactgaagtgaccgttcagctgatcaattataccaagagcggcaagaagttctggaacgtgttccacctgcagccgatgcgcgattataagggcgacgtccagtacttcattggcgtgcagctggatggcaccgaacgtcttcatggcgccgctgagcgtgaggcggtcatgctgatcaaaaagacagcctttcagattgctgaggcagcgaacgacgaaaattacttttaa

SEQ ID NO: 10 - protein, 336 aa, Artificial sequence
  Gluc-iLID
MHHHHHHKPTENNEDFNIVAVASNFATTDLDADRGKLPGKKLPLEVLKELEANARKAGCTRGCLICLSHIKCTPKMKKFIPGRCHTYEGDKESAQGGIGEAIVDIPEIPGFKDLEPLEQFIAQVDLCVDCTTGCLKGLANVQCSDLLKKWLPQRCATFASKIQGQVDKIKGAGGDGGGGSGGGGSGSGEFLATTLERIEKNFVITDPRLPDNPIIFASDSFLQLTEYSREEILGRNCRFLQGPETDRATVRKIRDAIDNQTEVTVQLINYTKSGKKFWNVFHLQPMRDYKGDVQYFIGVQLDGTERLHGAAEREAVMLIKKTAFQIAEAANDENYF

SEQ ID NO: 11 - DNA, 1233 bp, Artificial sequence
  Gluc-EL222
atgaaaccaacagaaaataacgaagattttaacatcgtcgcggtagcctcaaattttgcaaccaccgacttagacgcggatcgtgggaaattaccaggtaagaaattgccgttggaagtacttaaagaactggaagccaacgcacgcaaggctggatgcactcgtgggtgtttgatctgtctgtcgcatattaagtgtacgccaaagatgaaaaagttcatcccagggcgttgtcatacttacgaaggagacaaagagtcagcacagggcggaatcggggaagctatcgtagatatccctgagatcccaggcttcaaagacttggaaccgctggaacagtttattgctcaagttgacttgtgcgtcgactgtacgactgggtgccttaaagggctggcgaatgtccaatgtagtgatctgcttaagaagtggcttcctcagcgttgcgctacattcgcaagcaaaattcaggggcaagtggataagatcaaaggcgcgggtggtgacggtggtggtggttcaggtggtggtggttcaatgttggatatgggacaagatcggccgatcgatggaagtggggcacccggggcagacgacacacgcgttgaggtgcaaccgccggcgcagtgggtcctcgacctgatcgaggccagcccgatcgcatcggtcgtgtccgatccgcgtctcgccgacaatccgctgatcgccatcaaccaggccttcaccgacctgaccggctattccgaagaagaatgcgtcggccgcaattgccgattcctggcaggttccggcaccgagccgtggctgaccgacaagatccgccaaggcgtgcgcgagcacaagccggtgctggtcgagatcctgaactacaagaaggacggcacgccgttccgcaatgccgtgctcgttgcaccgatctacgatgacgacgacgagcttctctatttcctcggcagccaggtcgaagtcgacgacgaccagcccaacatgggcatggcgcgccgcgaacgcgccgcggaaatgctcaagacgctgtcgccgcgccagctcgaggttacgacgctggtggcatcgggcttgcgcaacaaggaagtggcggcccggctcggcctgtcggagaaaaccgtcaagatgcaccgcgggctggtgatggaaaagctcaacctgaagaccagcgccgatctggtgcgcattgccgtcgaagccggaatccatcatcatcatcatcactaa

SEQ ID NO: 12 - protein, 410 aa, Artificial sequence
  Gluc-EL222
MKPTENNEDFNIVAVASNFATTDLDADRGKLPGKKLPLEVLKELEANARKAGCTRGCLICLSHIKCTPKMKKFIPGRCHTYEGDKESAQGGIGEAIVDIPEIPGFKDLEPLEQFIAQVDLCVDCTTGCLKGLANVQCSDLLKKWLPQRCATFASKIQGQVDKIKGAGGDGGGGSGGGGSMLDMGQDRPIDGSGAPGADDTRVEVQPPAQWVLDLIEASPIASVVSDPRLADNPLIAINQAFTDLTGYSEEECVGRNCRFLAGSGTEPWLTDKIRQGVREHKPVLVEILNYKKDGTPFRNAVLVAPIYDDDDELLYFLGSQVEVDDDQPNMGMARRERAAEMLKTLSPRQLEVTTLVASGLRNKEVAARLGLSEKTVKMHRGLVMEKLNLKTSADLVRIAVEAGIHHHHHH

SEQ ID NO: 13 - protein, 11 aa, Artificial sequence
  cell penetrating peptide (CPP) sequence
YARAAARQARA

SEQ ID NO: 14 - protein, 7 aa, Artificial sequence
  cell penetrating peptide (CPP) sequence
YGRKKRR

SEQ ID NO: 15 - protein, 11 aa, Artificial sequence
  cell penetrating peptide (CPP) sequence
YGRKKRRQRRR

SEQ ID NO: 16 - protein, 5 aa, Artificial sequence
  cell penetrating peptide (CPP) sequence
RRQRR

SEQ ID NO: 17 - protein, 6 aa, Artificial sequence
  N terminal anchor amino acid sequence
CDQSSS

SEQ ID NO: 18 - protein, 6 aa, Artificial sequence
  anchor protein amino acid sequence
CDNSSS